SEQUENCE LISTING (22 sequences)

SEQ ID NO: 1 - protein, 448 aa, Artificial Sequence
  PA50-YTE heavy chain
QVQLVQSGAEVKKPGASVKVSCKASGYTFTDYNMDWVRQAPGQRLEWMGDINPKYDIIGHNPKFMGRVTITRDTSASTAYMELSSLRSEDTAVYYCARSDRGWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 211 aa, Artificial Sequence
  PA50-YTE Light Chain
EIVLTQSPATLSLSPGERATLSCRASSSVNYMNWYQQKPGQAPRPLIYATSNLASGIPARFSGSGSGTDFTLTISSLEPEDFAVYYCQQWSSRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 449 aa, Artificial Sequence
  PA41-YTE Heavy Chain
QVQLVQSGAEVKKPGASVKVSCKASGYPFTNYFMHWVRQAPGQRLEWIGRINPYNGATSYSLNFRDKATITLDKSASTAYMELSSLRSEDTAVYYCARSTITSPLLDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 214 aa, Artificial Sequence
  PA41-YTE Light Chain
EIVLTQSPATLSLSPGERATLSCRASQSVGTSIHWYQQKPGQAPRLLIKFASESISGIPARFSGSGSGTDFTLTISSLEPEDFAVYYCQQSNKWPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 2710 aa, Artificial Sequence
  Clostridium difficile toxin A (TcdA)
MSLISKEELIKLAYSIRPRENEYKTILTNLDEYNKLTTNNNENKYLQLKKLNESIDVFMNKYKTSSRNRALSNLKKDILKEVILIKNSNTSPVEKNLHFVWIGGEVSDIALEYIKQWADINAEYNIKLWYDSEAFLVNTLKKAIVESSTTEALQLLEEEIQNPQFDNMKFYKKRMEFIYDRQKRFINYYKSQINKPTVPTIDDIIKSHLVSEYNRDETVLESYRTNSLRKINSNHGIDIRANSLFTEQELLNIYSQELLNRGNLAAASDIVRLLALKNFGGVYLDVDMLPGIHSDLFKTISRPSSIGLDRWEMIKLEAIMKYKKYINNYTSENFDKLDQQLKDNFKLIIESKSEKSEIFSKLENLNVSDLEIKIAFALGSVINQALISKQGSYLTNLVIEQVKNRYQFLNQHLNPAIESDNNFTDTTKIFHDSLFNSATAENSMFLTKIAPYLQVGFMPEARSTISLSGPGAYASAYYDFINLQENTIEKTLKASDLIEFKFPENNLSQLTEQEINSLWSFDQASAKYQFEKYVRDYTGGSLSEDNGVDFNKNTALDKNYLLNNKIPSNNVEEAGSKNYVHYIIQLQGDDISYEATCNLFSKNPKNSIIIQRNMNESAKSYFLSDDGESILELNKYRIPERLKNKEKVKVTFIGHGKDEFNTSEFARLSVDSLSNEISSFLDTIKLDISPKNVEVNLLGCNMFSYDFNVEETYPGKLLLSIMDKITSTLPDVNKNSITIGANQYEVRINSEGRKELLAHSGKWINKEEAIMSDLSSKEYIFFDSIDNKLKAKSKNIPGLASISEDIKTLLLDASVSPDTKFILNNLKLNIESSIGDYIYYEKLEPVKNIIHNSIDDLIDEFNLLENVSDELYELKKLNNLDEKYLISFEDISKNNSTYSVRFINKSNGESVYVETEKEIFSKYSEHITKEISTIKNSIITDVNGNLLDNIQLDHTSQVNTLNAAFFIQSLIDYSSNKDVLNDLSTSVKVQLYAQLFSTGLNTIYDSIQLVNLISNAVNDTINVLPTITEGIPIVSTILDGINLGAAIKELLDEHDPLLKKELEAKVGVLAINMSLSIAATVASIVGIGAEVTIFLLPIAGISAGIPSLVNNELILHDKATSVVNYFNHLSESKKYGPLKTEDDKILVPIDDLVISEIDFNNNSIKLGTCNILAMEGGSGHTVTGNIDHFFSSPSISSHIPSLSIYSAIGIETENLDFSKKIMMLPNAPSRVFWWETGAVPGLRSLENDGTRLLDSIRDLYPGKFYWRFYAFFDYAITTLKPVYEDTNIKIKLDKDTRNFIMPTITTNEIRNKLSYSFDGAGGTYSLLLSSYPISTNINLSKDDLWIFNIDNEVREISIENGTIKKGKLIKDVLSKIDINKNKLIIGNQTIDFSGDIDNKDRYIFLTCELDDKISLIIEINLVAKSYSLLLSGDKNYLISNLSNIIEKINTLGLDSKNIAYNYTDESNNKYFGAISKTSQKSIIHYKKDSKNILEFYNDSTLEFNSKDFIAEDINVFMKDDINTITGKYYVDNNTDKSIDFSISLVSKNQVKVNGLYLNESVYSSYLDFVKNSDGHHNTSNFMNLFLDNISFWKLFGFENINFVIDKYFTLVGKTNLGYVEFICDNNKNIDIYFGEWKTSSSKSTIFSGNGRNVVVEPIYNPDTGEDISTSLDFSYEPLYGIDRYINKVLIAPDLYTSLININTNYYSNEYYPEIIVLNPNTFHKKVNINLDSSSFEYKWSTEGSDFILVRYLEESNKKILQKIRIKGILSNTQSFNKMSIDFKDIKKLSLGYIMSNFKSFNSENELDRDHLGFKIIDNKTYYYDEDSKLVKGLININNSLFYFDPIEFNLVTGWQTINGKKYYFDINTGAALISYKIINGKHFYFNNDGVMQLGVFKGPDGFEYFAPANTQNNNIEGQAIVYQSKFLTLNGKKYYFDNDSKAVTGWRIINNEKYYFNPNNAIAAVGLQVIDNNKYYFNPDTAIISKGWQTVNGSRYYFDTDTAIAFNGYKTIDGKHFYFDSDCVVKIGVFSTSNGFEYFAPANTYNNNIEGQAIVYQSKFLTLNGKKYYFDNNSKAVTGWQTIDSKKYYFNTNTAEAATGWQTIDGKKYYFNTNTAEAATGWQTIDGKKYYFNTNTAIASTGYTIINGKHFYFNTDGIMQIGVFKGPNGFEYFAPANTDANNIEGQAILYQNEFLTLNGKKYYFGSDSKAVTGWRIINNKKYYFNPNNAIAAIHLCTINNDKYYFSYDGILQNGYITIERNNFYFDANNESKMVTGVFKGPNGFEYFAPANTHNNNIEGQAIVYQNKFLTLNGKKYYFDNDSKAVTGWQTIDGKKYYFNLNTAEAATGWQTIDGKKYYFNLNTAEAATGWQTIDGKKYYFNTNTFIASTGYTSINGKHFYFNTDGIMQIGVFKGPNGFEYFAPANTHNNNIEGQAILYQNKFLTLNGKKYYFGSDSKAVTGLRTIDGKKYYFNTNTAVAVTGWQTINGKKYYFNTNTSIASTGYTIISGKHFYFNTDGIMQIGVFKGPDGFEYFAPANTDANNIEGQAIRYQNRFLYLHDNIYYFGNNSKAATGWVTIDGNRYYFEPNTAMGANGYKTIDNKNFYFRNGLPQIGVFKGSNGFEYFAPANTDANNIEGQAIRYQNRFLHLLGKIYYFGNNSKAVTGWQTINGKVYYFMPDTAMAAAGGLFEIDGVIYFFGVDGVKAPGIYG

SEQ ID NO: 6 - protein, 2366 aa, Artificial Sequence
  Clostridium difficile toxin B (TcdB)
MSLVNRKQLEKMANVRFRTQEDEYVAILDALEEYHNMSENTVVEKYLKLKDINSLTDIYIDTYKKSGRNKALKKFKEYLVTEVLELKNNNLTPVEKNLHFVWIGGQINDTAINYINQWKDVNSDYNVNVFYDSNAFLINTLKKTVVESAINDTLESFRENLNDPRFDYNKFFRKRMEIIYDKQKNFINYYKAQREENPELIIDDIVKTYLSNEYSKEIDELNTYIEESLNKITQNSGNDVRNFEEFKNGESFNLYEQELVERWNLAAASDILRISALKEIGGMYLDVDMLPGIQPDLFESIEKPSSVTVDFWEMTKLEAIMKYKEYIPEYTSEHFDMLDEEVQSSFESVLASKSDKSEIFSSLGDMEASPLEVKIAFNSKGIINQGLISVKDSYCSNLIVKQIENRYKILNNSLNPAISEDNDFNTTTNTFIDSIMAEANADNGRFMMELGKYLRVGFFPDVKTTINLSGPEAYAAAYQDLLMFKEGSMNIHLIEADLRNFEISKTNISQSTEQEMASLWSFDDARAKAQFEEYKRNYFEGSLGEDDNLDFSQNIVVDKEYLLEKISSLARSSERGYIHYIVQLQGDKISYEAACNLFAKTPYDSVLFQKNIEDSEIAYYYNPGDGEIQEIDKYKIPSIISDRPKIKLTFIGHGKDEFNTDIFAGFDVDSLSTEIEAAIDLAKEDISPKSIEINLLGCNMFSYSINVEETYPGKLLLKVKDKISELMPSISQDSIIVSANQYEVRINSEGRRELLDHSGEWINKEESIIKDISSKEYISFNPKENKITVKSKNLPELSTLLQEIRNNSNSSDIELEEKVMLTECEINVISNIDTQIVEERIEEAKNLTSDSINYIKDEFKLIESISDALCDLKQQNELEDSHFISFEDISETDEGFSIRFINKETGESIFVETEKTIFSEYANHITEEISKIKGTIFDTVNGKLVKKVNLDTTHEVNTLNAAFFIQSLIEYNSSKESLSNLSVAMKVQVYAQLFSTGLNTITDAAKVVELVSTALDETIDLLPTLSEGLPIIATIIDGVSLGAAIKELSETSDPLLRQEIEAKIGIMAVNLTTATTAIITSSLGIASGFSILLVPLAGISAGIPSLVNNELVLRDKATKVVDYFKHVSLVETEGVFTLLDDKIMMPQDDLVISEIDFNNNSIVLGKCEIWRMEGGSGHTVTDDIDHFFSAPSITYREPHLSIYDVLEVQKEELDLSKDLMVLPNAPNRVFAWETGWTPGLRSLENDGTKLLDRIRDNYEGEFYWRYFAFIADALITTLKPRYEDTNIRINLDSNTRSFIVPIITTEYIREKLSYSFYGSGGTYALSLSQYNMGINIELSESDVWIIDVDNVVRDVTIESDKIKKGDLIEGILSTLSIEENKIILNSHEINFSGEVNGSNGFVSLTFSILEGINAIIEVDLLSKSYKLLISGELKILMLNSNHIQQKIDYIGFNSELQKNIPYSFVDSEGKENGFINGSTKEGLFVSELPDVVLISKVYMDDSKPSFGYYSNNLKDVKVITKDNVNILTGYYLKDDIKISLSLTLQDEKTIKLNSVHLDESGVAEILKFMNRKGNTNTSDSLMSFLESMNIKSIFVNFLQSNIKFILDANFIISGTTSIGQFEFICDENDNIQPYFIKFNTLETNYTLYVGNRQNMIVEPNYDLDDSGDISSTVINFSQKYLYGIDSCVNKVVISPNIYTDEINITPVYETNNTYPEVIVLDANYINEKINVNINDLSIRYVWSNDGNDFILMSTSEENKVSQVKIRFVNVFKDKTLANKLSFNFSDKQDVPVSEIILSFTPSYYEDGLIGYDLGLVSLYNEKFYINNFGMMVSGLIYINDSLYYFKPPVNNLITGFVTVGDDKYYFNPINGGAASIGETIIDDKNYYFNQSGVLQTGVFSTEDGFKYFAPANTLDENLEGEAIDFTGKLIIDENIYYFDDNYRGAVEWKELDGEMHYFSPETGKAFKGLNQIGDYKYYFNSDGVMQKGFVSINDNKHYFDDSGVMKVGYTEIDGKHFYFAENGEMQIGVFNTEDGFKYFAHHNEDLGNEEGEEISYSGILNFNNKIYYFDDSFTAVVGWKDLEDGSKYYFDEDTAEAYIGLSLINDGQYYFNDDGIMQVGFVTINDKVFYFSDSGIIESGVQNIDDNYFYIDDNGIVQIGVFDTSDGYKYFAPANTVNDNIYGQAVEYSGLVRVGEDVYYFGETYTIETGWIYDMENESDKYYFNPETKKACKGINLIDDIKYYFDEKGIMRTGLISFENNNYYFNENGEMQFGYINIEDKMFYFGEDGVMQIGVFNTPDGFKYFAHQNTLDENFEGESINYTGWLDLDEKRYYFTDEYIAATGSVIIDGEEYYFDPDTAQLVISE

SEQ ID NO: 7 - protein, 117 aa, Artificial Sequence
  Anti-toxin A antibody, VH region of a humanized PA-39 (hPA-39)
QVQLVQSGAEVKKPGASVKVSCKASGYTFNDHNIHWVRQAPGQGLEWIGYIYPYIGTTVYNQKFKSKATLTVDTSTSTAYMELRSLRSDDTAVYYCSRWGHRGFPYWGQGTLVTVSS

SEQ ID NO: 8 - protein, 117 aa, Artificial Sequence
  Anti-toxin A antibody, VH region of a humanized PA-39 (hPA-39)
QVQLVQSGAEVKKPGASVKVSCKASGYTFNDHNIHWVRQAPGQGLEWIGYIYPYIGTTVYNQKFKSKATLTVDNSTSTAYMELRSLRSDDTAVYYCSRWGHRGFPYWGQGTLVTVSS

SEQ ID NO: 9 - protein, 107 aa, Artificial Sequence
  Anti-toxin A antibody, VL region of a humanized PA-39 (hPA-39)
DIQMTQSPSSLSASVGDRVTITCKASQNVGTNVAWYQQKPGKAPKALIYSASYRYSGVSSRFSGSGSGTDFTLTISSLQPEDFAVYYCQQYYSYPYTFGQGTKLEIK

SEQ ID NO: 10 - protein, 107 aa, Artificial Sequence
  Anti-toxin A antibody, VL region of a humanized PA-39 (hPA-39)
DIQMTQSPSSLSASVGDRVTITCKASQNVGTNVAWYQQKPGKAPKVLIYSASYRYSGVSSRFSGSGSGTDFTLTISSLQPEDFAVYYCQQYYSYPYTFGQGTKLEIK

SEQ ID NO: 11 - protein, 118 aa, Artificial Sequence
  Anti-toxin A antibody, VH region of a humanized PA-50 (hPA-50)
QVQLVQSGAEVKKPGASVKVSCKASGYTFTDYNMDWVRQAPGQRLEWIGDINPKYDIIGHNPKFMGKATLTVDKSASTAYMELSSLRSEDTAVYYCARSDRGWYFDVWGQGTLVTVSS

SEQ ID NO: 12 - protein, 118 aa, Artificial Sequence
  Anti-toxin A antibody, VH region of a humanized PA-50 (hPA-50)
QVQLVQSGAEVKKPGASVKVSCKASGYTFTDYNMDWVRQAPGQRLEWIGDINPKYDIIGHNPKFMGKATITVDKSASTAYMELSSLRSEDTAVYYCARSDRGWYFDVWGQGTLVTVSS

SEQ ID NO: 13 - protein, 104 aa, Artificial Sequence
  Anti-toxin A antibody, VL region of a humanized PA-50 (hPA-50)
EIVLTQSPATLSLSPGERATLSCRASSSVNYMNWYQQKPGQAPRPRIYATSNLASGVPARFSGSGSGTDYTLTISSLEPEDFAVYYCQQWSSRTFGGGTKVEIK

SEQ ID NO: 14 - protein, 119 aa, Artificial Sequence
  Anti-toxin B antibody, VH region of a humanized PA-41 (hPA-41)
QVQLVQSGAEVKKPGASVKVSCKASGYPFTNYFMHWVRQAPGQRLEWIGRINPYNGATSYSLNFRDKATLTLDKSASTAYMELSSLRSEDTAVYYCARSTITSPLLDFWGQGTLVTVSS

SEQ ID NO: 15 - protein, 119 aa, Artificial Sequence
  Anti-toxin B antibody, VH region of a humanized PA-41 (hPA-41)
QVQLVQSGAEVKKPGASVKVSCKASGYPFTNYFMHWVRQAPGQRLEWIGRINPYNGATSYSLNFRDKATITLDKSASTAYMELSSLRSEDTAVYYCARSTITSPLLDFWGQGTLVTVSS

SEQ ID NO: 16 - protein, 107 aa, Artificial Sequence
  Anti-toxin B antibody, VL region of a humanized PA-41 (hPA-41)
EIVLTQSPATLSLSPGERATLSCRASQSVGTSIHWYQQKPGQAPRLLIKFASESISGIPARFSGSGSGTDFTLTISSLEPEDFAVYYCQQSNKWPFTFGQGTKLEIK

SEQ ID NO: 17 - protein, 467 aa, Artificial Sequence
  Anti-toxin A antibody, heavy chain
MEWSGVFIFLLSVTAGVHSQVQLVQSGAEVKKPGASVKVSCKASGYTFTDYNMDWVRQAPGQRLEWIGDINPKYDIIGHNPKFMGKATITVDKSASTAYMELSSLRSEDTAVYYCARSDRGWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 233 aa, Artificial Sequence
  Anti-toxin A antibody, light chain
MDFQVQIFSFLLISASVIMSRGEIVLTQSPATLSLSPGERATLSCRASSSVNYMNWYQQKPGQAPRPRIYATSNLASGVPARFSGSGSGTDYTLTISSLEPEDFAVYYCQQWSSRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 538 aa, Artificial Sequence
  Anti-toxin A antibody, heavy chain
MEWSGVFIFLLSVTAGVHSQVQLVQSGAEVKKPGASVKVSCKASGYTFNDHNIHWVRQAPGQGLEWIGYIYPYIGTTVYNQKFKSKATLTVDTSTSTAYMELRSLRSDDTAVYYCSRWGHRGFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGERPAQGGRVSAGSQAQRSCLDASRLCSPSPGQQGRPRLPLHPEASARPTHAQGEGLLAFSPGSGQAQARCPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 234 aa, Artificial Sequence
  Anti-toxin A antibody, light chain
MESQTQVFVYMLLWLSGVDGDIQMTQSPSSLSASVGDRVTITCKASQNVGTNVAWYQQKPGKAPKALIYSASYRYSGVSSRFSGSGSGTDFTLTISSLQPEDFAVYYCQQYYSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 540 aa, Artificial Sequence
  Anti-toxin B antibody, heavy chain
MGWSWIFLFLLSGTAGGLSQVQLVQSGAEVKKPGASVKVSCKASGYPFTNYFMHWVRQAPGQRLEWIGRINPYNGATSYSLNFRDKATITLDKSASTAYMELSSLRSEDTAVYYCARSTITSPLLDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGERPAQGGRVSAGSQAQRSCLDASRLCSPSPGQQGRPRLPLHPEASARPTHAQGEGLLAFSPGSGQAQARCPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 234 aa, Artificial Sequence
  Anti-toxin B antibody, light chain
MSVPTQVLGLLLLWLTDARCEIVLTQSPATLSLSPGERATLSCRASQSVGTSIHWYQQKPGQAPRLLIKFASESISGIPARFSGSGSGTDFTLTISSLEPEDFAVYYCQQSNKWPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2018/0085458 A1
(43) Pub. Date: Mar. 29, 2018

(54) METHODS FOR TREATING CLOSTRIDIUM DIFFICILE INFECTION AND ASSOCIATED DISEASE

(71) Applicant: MedImmune, LLC, Gaithersburg, MD (US)

(72) Inventors: Andrew C NYBORG, Gaithersburg, MD (US); Paul WARRENER, Gaithersburg, MD (US); Godfrey RAINEY, Gaithersburg, MD (US)

(21) Appl. No.: 15/566,118

(22) PCT Filed: Apr. 14, 2016
(86) PCT No.: PCT/US16/27411

§ 371 Date: Oct. 12, 2017

Related U.S. Application Data
(60) Provisional application No. 62/147,908, filed on Apr. 15, 2015.

(51) Int. Cl. A61K 39/40; A61K 39/08; C07K 16/12; A61K 38/14

(52) U.S. Cl. CPC A61K 39/40; A61K 39/08; A61K 2039/507; A61K 38/14; C07K 16/1282

(57) ABSTRACT

The invention features methods for treating Clostridium difficile infection (CDI), C. difficile associated disease, and symptoms thereof, featuring the use of antibodies having enhanced half-life that specifically bind C. difficile toxin A and/or toxin B. In one aspect, the invention provides a method of treating a C. difficile infection or C. difficile-associated disease in a subject, the method involving administering to the subject a combination of an anti-C. difficile toxin A antibody and an anti-C. difficile toxin B antibody having an alteration that increases the half-life of one or both antibodies relative to anti-C. difficile toxin A and B antibodies lacking the alteration. In one aspect, the invention features a composition comprising an equimolar mixture of an anti-toxin A antibody and an anti-toxin B antibody. The invention provides kits for treating a C. difficile infection or symptoms thereof.

### BACKGROUND OF THE INVENTION

C. difficile infection (CDI), classified as an urgent public health threat by the Centers for Disease Control, is a bacterial toxin-mediated disease and a leading cause of hospital acquired infections. The majority of CDI is precipitated by intestinal microbiome dysbiosis (disruption of normal gut flora), a result of prior treatment with broad-spectrum antibiotics, which facilitates the proliferation of C. difficile. Paradoxically, the dysbiosis which allows this pathogen to cause disease is prolonged by the very antibiotics used to treat CDI, resulting in a high rate of disease recurrence.

Infection with Clostridium difficile, a Gram-positive spore-forming anaerobe, leads to symptoms that range from moderate diarrhea and pseudomembranous colitis to toxic megacolon, sepsis and death. C. difficile spores are resistant to most disinfectants and are shed into the hospital environment by both symptomatic patients and asymptomatic carriers. The annual rate of CDI has doubled since 2001, coincident with the emergence of hypervirulent strains. Over 500,000 new cases of C. difficile infection occur each year in the US and estimates suggest greater than 400,000 diagnosed CDI events occur annually in Europe. This represents a substantial burden of morbidity, mortality, and healthcare resource consumption that calls for a more effective treatment strategy.

CDI is most common in elderly patients with comorbidities—a fragile population—and infections are typically subsequent to treatment with broad-spectrum antibiotics. Antibiotic-mediated disruption of the beneficial intestinal microbiota allows colonization and infection with C. difficile. The antibiotics commonly used to treat CDI (metronidazole, vancomycin and fidaxomicin) prolong intestinal dysbiosis and lead to a 13-25% rate of infection recurrence following cessation of antibiotic therapy. A lasting cure for CDI requires the restoration of a diverse and protective intestinal microbiome that is resistant to infection recurrence. Indeed, it has been advances in understanding of C. difficile pathogenesis and resistance that have helped clarify the important role of the beneficial gut microbiome in maintaining overall health.

At present, effective treatments and preventatives for C. difficile infection and illness are lacking. New methods of treatment are urgently required.

### SUMMARY OF THE INVENTION

As described below, the invention generally features methods for treating C. difficile infection (CDI), C. difficile associated disease, and symptoms thereof, featuring the use of antibodies having enhanced half-life that specifically bind C. difficile toxin A and/or toxin B.

In one aspect, the invention provides a method of treating a C. difficile infection or C. difficile-associated disease in a subject, the method involving administering to the subject a combination of an anti-C. difficile toxin A antibody and an anti-C. difficile toxin B antibody having an alteration that increases the half-life of one or both antibodies relative to anti-C. difficile toxin A and B antibodies lacking the alteration.

In another aspect, the invention provides a method of treating a C. difficile infection or C. difficile-associated disease in a subject, the method involving administering to the subject a combination of an anti-C. difficile toxin A antibody and an anti-C. difficile toxin B antibody and vancomycin, to thereby reduce the dose or dose frequency of vancomycin relative to a reference dose or dose frequency.

In various embodiments of any aspect delineated herein, one or both antibodies have increased half-life relative to anti-C. difficile toxin A and B antibodies lacking the alteration. In certain embodiments, the alteration is any one or more of 252Y, 254T, or 256E (e.g., YTE modification). In some embodiments, the alteration is conjugation to polyethylene glycol (PEG) or conjugation to albumin.

In various embodiments of any aspect delineated herein, the anti-toxin A antibody has a heavy chain containing the sequence SEQ ID NO: 1:

qvqlvqsgaevkkpgasvkvsckasgytftdynmdwvrqapgqrlewmgd
inpkydiighnpkfmgrvtitrdtsastaymelsslrsedtavyycarsd
rgwyfdvwgqgtlvtvssastkgpsvfplapsskstsggtaalgclvkdy
fpepvtvswnsgaltsgvhtfpavlqssglyslssvvtvpssslgtqtyi
cnvnhkpsntkvdkrvepkscdkthtcppcpapellggpsvflfppkpkd
tlyitrepevtcvvvdvshedpevkfnwyvdgvevhnaktkpreeqynst
yrvvsyltvlhqdwlngkeykckvsnkalpapiektiskakgqprepqvy
tlppsreemtknqvsltclvkgfypsdiavewesngqpennykttppvld
sdgsfflyskltvdksrwqqgnvfscsvmhealhnhytqkslslspgk.

In various embodiments of any aspect delineated herein, the anti-toxin A antibody has a light chain containing the sequence SEQ ID NO: 2:

eivltqspatlslspgeratlscrasssvnymnwyqqkpgqaprpliyat
snlasgiparfsgsgsgtdftltisslepedfavyycqqwssrtfgggtk
leikrtvaapsvfifppsdeqlksgtasvvcllnnfypreakvqwkvdna
lqsgnsqesvteqdskdstyslsstltlskadyekhkvyacevthqglss
pvtksfnrgec.

In various embodiments of any aspect delineated herein, the anti-toxin B antibody has a heavy chain containing the sequence SEQ ID NO: 3:

qvqlvqsgaevkkpgasvkvsckasgypftnyfmhwvrqapgqrlewigr
inpyngatsyslnfrdkatitldksastaymelsslrsedtavyycarst
itsplldfwgqgtlvtvssastkgpsvfplapsskstsggtaalgclvkd
yfpepvtvswnsgaltsgvhtfpavlqssglyslssvvtvpssslgtqty
icnvnhkpsntkvdkrvepkscdkthtcppcpapellggpsvflfppkpk
dtlyitrepevtcvvvdvshedpevkfnwyvdgvevhnaktkpreeqyns
tyrvvsvltvlhqdwlngkeykckvsnkalpapiektiskakgqprepqv
ytlppsreemtknqvsltclvkgfypsdiavewesngqpennykttppvl
dsdgsfflyskltvdksrwqqgnvfscsvmhealhnhytqkslslspgk.

In various embodiments of any aspect delineated herein, the anti-toxin B antibody has a

eivltqspatlslspgeratlscrasqsvgtsihwyqqkpgqaprllikf
asesisgiparfsgsgsgtdftltisslepedfavyycqqsnkwpftfgq
gtkleikrtvaapsvfifppsdeqlksgtasvvcllnnfypreakvqwkv
dnalqsgnsqesvteqdskdstyslsstltlskadyekhkvyacevthqg
lsspvtksfnrgec.

In various embodiments, the anti-toxin A antibody is PA50-YTE. In various embodiments, the anti-toxin B antibody is PA41-YTE. In particular embodiments, the combination of the antibodies is PA50YTE/PA41YTE COMBINATION. In certain embodiments, PA50YTE/PA41YTE COMBINATION is administered in a single dose.

In further embodiments of any aspect delineated herein, the method of treatment further involves administering an antibiotic, such as vancomycin, fidaxomicin and metronidazole. In various embodiments, the antibiotic is administered orally or intravenously.

In various embodiments of any aspect delineated herein, the method of treatment further involves administering vancomycin. In various embodiments, the vancomycin is administered orally or intravenously. In certain embodiments, the reference dose and dose frequency is intravenous administration of vancomycin at 15-20 mg/kg, 2-3 times daily. In some embodiments, the reference dose and dose frequency is oral administration at 125 mg, 3-4 times daily.

In various embodiments of any aspect delineated herein, C. difficile toxin A and/or toxin B are neutralized. In various embodiments of any aspect delineated herein, the method of treatment reduces the time to C. difficile reinfection. In various embodiments of any aspect delineated herein, the method of treatment enhances microbiome restoration, reduces microbiome dysbiosis, and/or reduces intestinal damage in the subject, including for example, relative to an antibiotic therapy.

Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “Clostridium difficile toxin A (TcdA)” is meant a polypeptide or fragment thereof having at least about 85% or greater amino acid identity to the amino acid sequence provided at NCBI Accession No. YP_001087137 and having TcdA biological activity. TcdA biological activity includes glucosylating activity, such as glucosylation of GTPases (e.g., Rho, Rac, and Cdc42). An exemplary C. difficile toxin A sequence is provided below (SEQ ID NO: 5):

| | |
|---|---|
| 1 | msliskeeli klaysirpre neyktiltnl deynklttnn nenkylqlkk lnesidvfmn |
| 61 | kyktssrnra lsnlkkdilk eviliknsnt spveknlhfv wiggevsdia leyikqwadi |
| 121 | naeyniklwy dseaflvntl kkaivesstt ealqlleeei qnpqfdnmkf ykkrmefiyd |
| 181 | rqkrfinyyk sqinkptvpt iddiikshlv seynrdetvl esyrtnslrk insnhgidir |
| 241 | anslfteqel lniysqelln rgnlaaasdi vrllalknfg gvyldvdmlp gihsdlfkti |
| 301 | srpssigldr wemikleaim kykkyinnyt senfdkldqg lkdnfkliie sksekseifs |
| 361 | klenlnvsdl eikiafalgs vinqaliskq gsyltnlvie qvknryqfln qhlnpaiesd |
| 421 | nnftdttkif hdslfnsata ensmfltkia pylqvgfmpe arstislsgp gayasayydf |
| 481 | inlqentiek tlkasdlief kfpennlsql teqeinslws fdqasakyqf ekyvrdytgg |
| 541 | slsedngvdf nkntaldkny llnnkipsnn veeagsknyv hyiiqlqgdd isyeatcnlf |
| 601 | sknpknsiii qrnmnesaks yflsddgesi lelnkyripe rlknkekvkv tfighgkdef |
| 661 | ntsefarlsv dslsneissf ldtikldisp knvevnllgc nmfsydfnve etypgkllls |
| 721 | imdkitstlp dvnknsitig anqyevrins egrkellahs gkwinkeeai msdlsskeyi |
| 781 | ffdsidnklk aksknipgla sisediktll ldasvspdtk filnnlklni essigdyiyy |
| 841 | eklepvknii hnsiddlide fnllenvsde lyelkklnnl dekylisfed isknnstysv |
| 901 | rfinksnges vyvetekeif skysehitke istiknsiit dvngnlldni qldhtsqvnt |
| 961 | lnaaffiqsl idyssnkdvl ndlstsvkvq lyaqlfstgl ntiydsiqlv nlisnavndt |
| 1021 | invlptiteg ipivstildg inlgaaikel ldehdpllkk eleakvgvla inmslsiaat |
| 1081 | vasivgigae vtifllpiag isagipslvn nelilhdkat svvnyfnhls eskkygplkt |
| 1141 | eddkilvpid dlviseidfn nnsiklgtcn ilameggsgh tvtgnidhff sspsisship |
| 1201 | slsiysaigi etenldfskk immlpnapsr vfwwetgavp glrslendgt rlldsirdly |
| 1261 | pgkfywrfya ffdyaittlk pvyedtniki kldkdtrnfi mptittneir nklsysfdga |
| 1321 | ggtyslllss ypistninls kddlwifnid nevreisien gtikkgklik dvlskidink |
| 1381 | nkliignqti dfsgdidnkd ryifltceld dkisliiein lvaksyslll sgdknylisn |
| 1441 | lsniiekint lgldskniay nytdesnnky fgaisktsqk siihykkdsk nilefyndst |
| 1501 | lefnskdfia edinvfmkdd intitgkyyv dnntdksidf sislvsknqv kvnglylnes |
| 1561 | vyssyldfvk nsdghhntsn fmnlfldnis fwklfgfeni nfvidkyftl vgktnlgyve |
| 1621 | ficdnnknid iyfgewktss skstifsgng rnvvvepiyn pdtgedists ldfsyeplyg |
| 1681 | idryinkvli apdlytslin intnyysney ypeiivlnpn tfhkkvninl dsssfeykws |
| 1741 | tegsdfilvr yleesnkkil qkirikgils ntqsfnkmsi dfkdikklsl gyimsnfksf |
| 1801 | nseneldrdh lgfkiidnkt yyydedsklv kglininnsl fyfdpiefnl vtgwqtingk |
| 1861 | kyyfdintga alisykiing khfyfnndgv mqlgvfkgpd gfeyfapant qnnniegqai |
| 1921 | vyqskfltln gkkyyfdnds kavtgwriin nekyyfnpnn aiaavglqvi dnnkyyfnpd |
| 1981 | taiiskgwqt vngsryyfdt dtaiafngyk tidgkhfyfd sdcvvkigvf stsngfeyfa |
| 2041 | pantynnnie gqaivyqskf ltlngkkyyf dnnskavtgw qtidskkyyf ntntaeaatg |
| 2101 | wqtidgkkyy fntntaeaat gwqtidgkky yfntntaias tgytiingkh fyfntdgimq |
| 2161 | igvfkgpngf eyfapantda nniegqaily qnefltlngk kyyfgsdska vtgwriinnk |
| 2221 | kyyfnpnnai aaihlctinn dkyyfsydgi lqngyitier nnfyfdanne skmvtgvfkg |
| 2281 | pngfeyfapa nthnnniegq aivyqnkflt lngkkyyfdn dskavtgwqt idgkkyyfnl |
| 2341 | ntaeaatgwq tidgkkyyfn lntaeaatgw qtidgkkyyf ntntfiastg ytsingkhfy |
| 2401 | fntdgimqig vfkgpngfey fapanthnnn iegqailyqn kfltlngkky yfgsdskavt |
| 2461 | glrtidgkky yfntntavav tgwqtingkk yyfntntsia stgytiisgk hfyfntdgim |
| 2521 | qigvfkgpdg feyfapantd anniegqair yqnrflylhd niyyfgnnsk aatgwvtidg |
| 2581 | nryyfepnta mgangyktid nknfyfrngl pqigvfkgsn gfeyfapant danniegqai |
| 2641 | rygnrflhll gkiyyfgnns kavtgwqtin gkvyyfmpdt amaaagglfe idgviyffgv |
| 2701 | dgvkapgiyg |

By “Clostridium difficile toxin B (TcdB)” is meant a polypeptide or fragment thereof having at least about 85% or greater amino acid identity to the amino acid sequence provided at NCBI Accession No. YP_001087135 and having TcdB biological activity. TcdB biological activity includes glucosylating activity, such as glucosylation of GTPases (e.g., Rho, Rac, and Cdc42). An exemplary C. difficile toxin B sequence is provided below (SEQ ID NO: 6):

| | |
|---|---|
| 1 | mslvnrkqle kmanvrfrtq edeyvailda leeyhnmsen tvvekylklk dinsltdiyi |
| 61 | dtykksgrnk alkkfkeylv tevlelknnn ltpveknlhf vwiggqindt ainyinqwkd |
| 121 | vnsdynvnvf ydsnaflint lkktvvesai ndtlesfren lndprfdynk ffrkrmeiiy |
| 181 | dkqknfinyy kaqreenpel iiddivktyl sneyskeide lntyieesln kitqnsgndv |
| 241 | rnfeefknge sfnlyeqelv erwnlaaasd ilrisalkei ggmyldvdml pgiqpdlfes |
| 301 | iekpssvtvd fwemtkleai mkykeyipey tsehfdmlde evqssfesvl asksdkseif |
| 361 | sslgdmeasp levkiafnsk giinqglisv kdsycsnliv kqienrykil nnslnpaise |
| 421 | dndfntttnt fidsimaean adngrfmmel gkylrvgffp dvkttinlsg peayaaayqd |
| 481 | llmfkegsmn ihlieadlrn feisktnisq steqemaslw sfddarakaq feeykrnyfe |
| 541 | gslgeddnld fsqnivvdke yllekissla rssergyihy ivqlqgdkis yeaacnlfak |
| 601 | tpydsvlfqk niedseiayy ynpgdgeiqe idkykipsii sdrpkikltf ighgkdefnt |
| 661 | difagfdvds lsteieaaid lakedispks ieinllgcnm fsysinveet ypgklllkvk |
| 721 | dkiselmpsi sqdsiivsan qyevrinseg rrelldhsge winkeesiik disskeyisf |
| 781 | npkenkitvk sknlpelstl lqeirnnsns sdieleekvm lteceinvis nidtqiveer |
| 841 | ieeaknltsd sinyikdefk liesisdalc dlkqqneled shfisfedis etdegfsirf |
| 901 | inketgesif vetektifse yanhiteeis kikgtifdtv ngklvkkvnl dtthevntln |
| 961 | aaffiqslie ynsskeslsn lsvamkvqvy aqlfstglnt itdaakvvel vstaldetid |
| 1021 | llptlseglp iiatiidgvs lgaaikelse tsdpllrqei eakigimavn lttattaiit |
| 1081 | sslgiasgfs illvplagis agipslvnne lvlrdkatkv vdyfkhvslv etegvftlld |
| 1141 | dkimmpqddl viseidfnnn sivlgkceiw rmeggsghtv tddidhffsa psityrephl |
| 1201 | siydvlevqk eeldlskdlm vlpnapnrvf awetgwtpgl rslendgtkl ldrirdnyeg |
| 1261 | efywryfafi adalittlkp ryedtnirin ldsntrsfiv piitteyire klsysfygsg |
| 1321 | gtyalslsqy nmginielse sdvwiidvdn vvrdvtiesd kikkgdlieg ilstlsieen |
| 1381 | kiilnshein fsgevngsng fvsltfsile ginaiievdl lsksykllis gelkilmlns |
| 1441 | nhiqqkidyi gfnselqkni pysfvdsegk engfingstk eglfvselpd vvliskvymd |
| 1501 | dskpsfgyys nnlkdvkvit kdnvniltgy ylkddikisl sltlqdekti klnsvhldes |
| 1561 | gvaeilkfmn rkgntntsds lmsflesmni ksifvnflqs nikfildanf iisgttsigq |
| 1621 | feficdendn iqpyfikfnt letnytlyvg nrqnmivepn ydlddsgdis stvinfsqky |
| 1681 | lygidscvnk vvispniytd einitpvyet nntypevivl danyinekin vnindlsiry |
| 1741 | vwsndgndfi lmstseenkv sqvkirfvnv fkdktlankl sfnfsdkqdv pvseiilsft |
| 1801 | psyyedglig ydlglvslyn ekfyinnfgm mvsgliyind slyyfkppvn nlitgfvtvg |
| 1861 | ddkyyfnpin ggaasigeti iddknyyfnq sgvlqtgvfs tedgfkyfap antldenleg |
| 1921 | eaidftgkli ideniyyfdd nyrgavewke ldgemhyfsp etgkafkgln qigdykyyfn |
| 1981 | sdgvmqkgfv sindnkhyfd dsgvmkvgyt eidgkhfyfa engemqigvf ntedgfkyfa |
| 2041 | hhnedlgnee geeisysgil nfnnkiyyfd dsftavvgwk dledgskyyf dedtaeayig |
| 2101 | lslindgqyy fnddgimqvg fvtindkvfy fsdsgiiesg vqniddnyfy iddngivqig |
| 2161 | vfdtsdgyky fapantvndn iygqaveysg lvrvgedvyy fgetytietg wiydmenesd |
| 2221 | kyyfnpetkk ackginlidd ikyyfdekgi mrtglisfen nnyyfnenge mqfgyinied |
| 2281 | kmfyfgedgv mqigvfntpd gfkyfahqnt ldenfegesi nytgwldlde kryyftdeyi |
| 2341 | aatgsviidg eeyyfdpdta qlvise |

The term “half-life” or “in vivo half-life” as used herein refers to a biological half-life of an antibody (e.g., IgG), or a fragment thereof, containing FcRn-binding sites in the circulation of a given animal and is represented by a time required for half the quantity administered in the animal to be cleared from the circulation and/or other tissues in the animal. When a clearance curve of a given IgG is constructed as a function of time, the curve is usually biphasic with a rapid α-phase which represents an equilibration of the injected IgG molecules between the intra- and extra-vascular space and which is, in part, determined by the size of molecules, and a longer β-phase which represents the catabolism of the IgG molecules in the intravascular space. The term “in vivo half-life” practically corresponds to the half-life of the IgG molecules in the β-phase.

By “antibody having increased half-life” is meant an antibody having increased biological half-life when compared to a reference antibody. In particular embodiments, the reference antibody is an antibody that lacks an alteration or modification (e.g., an unmodified parent or precursor antibody).

By “anti-tcdA antibody” is meant an antibody that specifically binds C. difficile toxin A. Anti-tcdA antibodies include monoclonal and polyclonal antibodies that are specific for C. difficile toxin A, and antigen-binding fragments thereof. In certain aspects, anti-tcdA antibodies as described herein are monoclonal antibodies (or antigen-binding fragments thereof), e.g., murine, humanized, or fully human monoclonal antibodies, including modified derivatives thereof. Exemplary anti-tcdA antibodies (e.g., PA-50, PA-39, and PA-38) are described in US20130202618/U.S. Pat. No. 8,986,697, which are incorporated herein by reference in their entireties. In one particular embodiment, the anti-tcdA antibody is PA50-YTE, which has the following heavy and light chain sequences:

PA50-YTE Light Chain (SEQ ID NO: 2):
eivltqspatlslspgeratlscrasssvnymnwyqqkpgqaprpliyat
snlasgiparfsgsgsgtdftltisslepedfavyycqqwssrtfgggtk
leikrtvaapsvfifppsdeqlksgtasvvcllnnfypreakvqwkvdna
lqsgnsqesvteqdskdstyslsstltlskadyekhkvyacevthqglss
pvtksfnrgec
PA50-YTE Heavy Chain (SEQ ID NO: 1):
qvqlvqsgaevkkpgasvkvsckasgytftdynmdwvrqapgqrlewmgd
inpkydiighnpkfmgrvtitrdtsastaymelsslrsedtavyycarsd
rgwyfdvwgqgtlvtvssastkgpsvfplapsskstsggtaalgclvkdy
fpepvtvswnsgaltsgvhtfpavlqssglyslssvvtvpssslgtqtyi
cnvnhkpsntkvdkrvepkscdkthtcppcpapellggpsvflfppkpkd
tlyitrepevtcvvvdvshedpevkfnwyvdgvevhnaktkpreeqynst
yrvvsvltvlhqdwlngkeykckvsnkalpapiektiskakgqprepqvy
tlppsreemtknqvsltclvkgfypsdiavewesngqpennykttppvld
sdgsfflyskltvdksrwqqgnvfscsvmhealhnhytqkslslspgk

By “anti-tcdB antibody” is meant an antibody that specifically binds C. difficile toxin B. Anti-tcdB antibodies include monoclonal and polyclonal antibodies that are specific for C. difficile toxin B, and antigen-binding fragments thereof. In certain aspects, anti-tcdB antibodies as described herein are monoclonal antibodies (or antigen-binding fragments thereof), e.g., murine, humanized, or fully human monoclonal antibodies, including modified derivatives thereof. Exemplary anti-tcdB antibodies (e.g., PA-41) are described in US20130202618/U.S. Pat. No. 8,986,697, which are incorporated herein by reference in their entireties. In one particular embodiment, the anti-tcdB antibody is PA41-YTE, which has the following heavy and light chain sequences:

PA41-YTE Light Chain
(SEQ ID NO: 4)
eivltqspatlslspgeratlscrasqsvgtsihwyqqkpgqaprllikf
asesisgiparfsgsgsgtdftltisslepedfavyycqqsnkwpftfgq
gtkleikrtvaapsvfifppsdeqlksgtasvvcllnnfypreakvqwkv
dnalqsgnsqesvteqdskdstyslsstltlskadyekhkvyacevthqg
lsspvtksfnrgec
PA41-YTE Heavy Chain
(SEQ ID NO: 3)
qvqlvqsgaevkkpgasvkvsckasgypftnyfmhwvrqapgqrlewigr
inpyngatsyslnfrdkatitldksastaymelsslrsedtavyycarst
itsplldfwgqgtlvtvssastkgpsvfplapsskstsggtaalgclvkd
yfpepvtvswnsgaltsgvhtfpavlqssglyslssvvtvpssslgtqty
icnvnhkpsntkvdkrvepkscdkthtcppcpapellggpsvflfppkpk
dtlyitrepevtcvvvdvshedpevkfnwyvdgvevhnaktkpreeqyns
tyrvvsvltvlhqdwlngkeykckvsnkalpapiektiskakgqprepqv
ytlppsreemtknqvsltclvkgfypsdiavewesngqpennykttppvl
dsdgsfflyskltvdksrwqqgnvfscsvmhealhnhytqkslslspgk

By “ameliorate” is meant decrease, suppress, attenuate, diminish, arrest, or stabilize the development or progression of a disease.

The term “antibody,” as used in this disclosure, refers to an immunoglobulin or a fragment or a derivative thereof, and encompasses any polypeptide comprising an antigen-binding site, regardless of whether it is produced in vitro or in vivo. The term includes, but is not limited to, polyclonal, monoclonal, monospecific, polyspecific, non-specific, humanized, single-chain, chimeric, synthetic, recombinant, hybrid, mutated, and grafted antibodies. Unless otherwise modified by the term “intact,” as in “intact antibodies,” for the purposes of this disclosure, the term “antibody” also includes antibody fragments such as Fab, F(ab′)2, Fv, scFv, Fd, dAb, and other antibody fragments that retain antigen-binding function, i.e., the ability to bind a C. difficile toxin A or toxin B polypeptide specifically. Typically, such fragments would comprise an antigen-binding domain.

The terms “antigen-binding domain,” “antigen-binding fragment,” and “binding fragment” refer to a part of an antibody molecule that comprises amino acids responsible for the specific binding between the antibody and the antigen. In instances, where an antigen is large, the antigen-binding domain may only bind to a part of the antigen. A portion of the antigen molecule that is responsible for specific interactions with the antigen-binding domain is referred to as “epitope” or “antigenic determinant.” In particular embodiments, an antigen-binding domain comprises an antibody light chain variable region (V_{L}) and an antibody heavy chain variable region (V_{H}), however, it does not necessarily have to comprise both. For example, a so-called Fd antibody fragment consists only of a V_{H} domain, but still retains some antigen-binding function of the intact antibody.

Binding fragments of an antibody are produced by recombinant DNA techniques, or by enzymatic or chemical cleavage of intact antibodies. Binding fragments include Fab, Fab′, F(ab′)2, Fv, and single-chain antibodies. An antibody other than a “bispecific” or “bifunctional” antibody is understood to have each of its binding sites identical. Digestion of antibodies with the enzyme, papain, results in two identical antigen-binding fragments, known also as “Fab” fragments, and a “Fc” fragment, having no antigen-binding activity but having the ability to crystallize. Digestion of antibodies with the enzyme, pepsin, results in the a F(ab′)2 fragment in which the two arms of the antibody molecule remain linked and comprise two-antigen binding sites. The F(ab′)2 fragment has the ability to crosslink antigen. “Fv” when used herein refers to the minimum fragment of an antibody that retains both antigen-recognition and antigen-binding sites. “Fab” when used herein refers to a fragment of an antibody that comprises the constant domain of the light chain and the CHI domain of the heavy chain.

The term “mAb” refers to monoclonal antibody. Antibodies of the invention comprise without limitation whole native antibodies, bispecific antibodies; chimeric antibodies; Fab, Fab′, single chain V region fragments (scFv), fusion polypeptides, and unconventional antibodies.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “C. difficile-associated disease” is meant any disease or symptom thereof associated with a C. difficile infection. C. difficile-associated diseases are characterized by one or more of the following symptoms: diarrhea, pseudomembranous colitis, toxic megacolon, perforation of the colon, and, in some instances, sepsis.

The term “effective amount” refers to a dosage or amount of an agent that is sufficient to reduce or stabilize a C. difficile infection in a subject or to reduce and/or ameliorate symptoms associated with a C. difficile infection in a patient or to otherwise achieve a desired biological outcome.

As used herein, “neutralize” refers to the reduction, inhibition, blocking, amelioration, or elimination of adverse effect(s) of the toxin(s) which the antibody(ies) specifically bind. Neutralization of adverse effect(s) of the toxin(s) includes 1) delaying, reducing, inhibiting, or preventing onset or progression of C. difficile infection or C. difficile-associated diarrhea or disease, 2) increasing survival of a subject as compared to the median survival of subjects who have not been treated with the antibody(ies) and who have C. difficile infection or C. difficile-associated disease, 3) eliminating one or more symptoms or adverse effects or reducing the severity of one or more symptoms or adverse effects associated with C. difficile infection or C. difficile-associated diarrhea or disease, 4) allowing for the repopulation of the normal microflora of the gastrointestinal tract of subjects who are or have been infected with C. difficile, 5) preventing a recurrence of C. difficile infection or C. difficile-associated disease in subjects who have been afflicted with C. difficile infection or C. difficile-associated disease, 6) effecting a cure rate of at least 50%, 55%, 60%, 65%, 70%, 75%, 80%, 85%, 90%, 95%, 97%, 98%, 99%, or 100% in subjects who have C. difficile infection or C. difficile-associated disease, and/or 7) preventing death due to CDAD or other adverse events associated with C. difficile infection.

The term “isolated” refers to a molecule that is substantially free of other elements present in its natural environment. For instance, an isolated protein is substantially free of cellular material or other proteins from the cell or tissue source from which it is derived. The term “isolated” also refers to preparations where the isolated protein is sufficiently pure to be administered as a pharmaceutical composition, or at least 70-80% (w/w) pure, more preferably, at least 80-90% (w/w) pure, even more preferably, 90-95% pure; and, most preferably, at least 95%, 96%, 97%, 98%, 99%, or 100% (w/w) pure.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. In a particular embodiment, a fragment of a polypeptide may contain 5, 10, 20, 30, 40, 50, 60, 70, 80, 90, 100, 200, or 300 amino acids.

By “reference” is meant a standard of comparison.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “specifically binds” is meant an agent (e.g., antibody) that recognizes and binds a molecule (e.g., polypeptide), but which does not substantially recognize and bind other molecules in a sample, for example, a biological sample. For example, two molecules that specifically bind form a complex that is relatively stable under physiologic conditions. Specific binding is characterized by a high affinity and a low to moderate capacity as distinguished from nonspecific binding which usually has a low affinity with a moderate to high capacity.

Typically, binding is considered specific when the affinity constant K_{A} is higher than 10⁷ M⁻¹, or more preferably higher than 10⁸ M⁻¹.

By “subject” is meant a mammal, including, but not limited to, a human or non-human mammal, such as a bovine, equine, canine, ovine, feline, or murine.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 shows that PA50YTE/PA41YTE COMBINATION, a combination of anti-toxin A and anti-toxin B monoclonal antibodies having enhanced half-life, provided a superior post infection protective benefit relative to antibiotic treatment in a C. difficile hamster infection model. A graph depicts the survival results of the groups of animals of the study. As depicted in the schematic, animals were challenged with C. difficile spores orally at day 0 of the study and were treated with clindamycin (10 mg/kg) at day 1. Study groups included infected control animals receiving no treatment, animals treated with vancomycin, and animals treated with a combination of murine anti-toxin A and anti-toxin B monoclonal antibodies having enhanced half-life. Animals treated with a combination of the anti-toxin A and anti-toxin B monoclonal antibodies survived and were protected against C. difficile toxicity for the duration of the study.

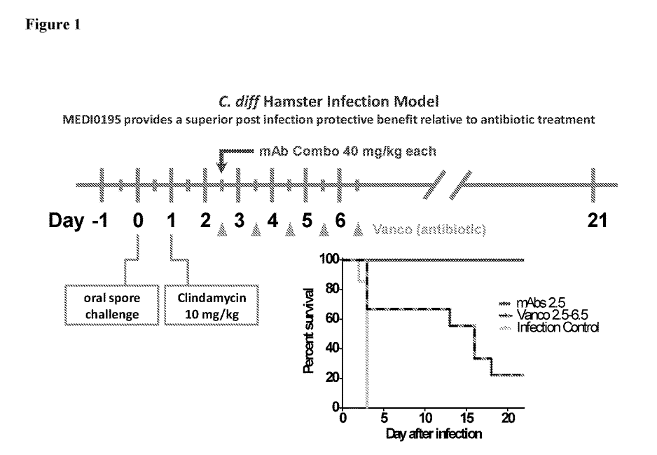

### DETAILED DESCRIPTION OF THE INVENTION

The invention features methods for treating C. difficile infection (CDI), C. difficile associated disease, and symptoms thereof, featuring antibodies having enhanced half-life that specifically bind C. difficile toxin A and/or toxin B.

The present invention is based, at least in part, on the discovery that a mixture of two monoclonal antibodies (mAbs) having increased half-life, neutralizes C. difficile toxins A and B, the key virulence factors of this pathogen. This combination represents a pathogen-focused, precision medicine alternative to antibiotic therapy. In preclinical survival models, toxin neutralization by such a combination was at least as effective, if not more effective, than antibiotics in treating CDI. By attacking these virulence factors directly, this treatment has the potential for more rapid resolution of symptoms while allowing patients to restore their CDI-resistant microbiome sooner than would be possible with current standard of care antibiotic therapy Such combinations have the added benefit of providing long-term neutralization of toxins A and B thereby further reducing the potential for recurrence. Treatment of C. difficile infections with such combinations supports the goals of advancing antibiotic stewardship and accelerating recovery from antibiotic-mediated microbiome dysbiosis, the underlying risk factor for CDI. Ongoing and proposed preclinical studies aim to demonstrate the impact of such combinations on microbiome restoration and the extent of intestinal damage, providing evidence for additional benefit over current antibiotic treatments.

C. difficile Infection (CDI) and C. difficile-Associated Disease (CDAD)

C. difficile-associated disease (CDAD) typically is precipitated by the disruption of the colonic flora through the use of antibiotics such as clindamycin, cephalosporins, and fluoroquinolones. This perturbation in the colonic microenvironment, along with exposure to C. difficile spores, leads to colonization in afflicted individuals. Approximately one-third of all patients who become colonized develop CDAD, which can result in severe diarrhea, perforation of the colon, colectomy and death. Methods, therefore, are provided whereby a subject is administered one or more antibodies of the invention to treat C. difficile infection or CDAD.

As used herein, to “treat” refers to any benefit to a subject with C. difficile infection or C. difficile-associated disease conferred through the administration of the antibodies and therapies provided herein. For example and without limitation, such a benefit can be the elimination of one or more symptoms or adverse effects, or a reduction in, or amelioration of, the severity of the one or more symptoms or adverse effects that result from the infection or disease; a delay, halt, or reversal in the progression of the infection or disease; a recolonization, resurgence, or repopulation of the normal and natural microflora of the gastrointestinal tract, colon, bowel, etc., or the cure of the infection or disease (i.e., a clinician would evaluate the subject and determine that the subject no longer has the infection or disease). Symptoms or adverse effects associated with C. difficile infection include dehydration, diarrhea, cramping, kidney failure, bowel perforation, toxic megacolon, which can lead to rupture of the colon, and death. The therapeutic methods provided can be used to reduce, diminish, ameliorate, or eliminate any or all of the symptoms or adverse effects provided herein.

As used herein, a “C. difficile infection” refers to an infection that results from the presence of C. difficile in the intestinal flora where it was not previously present or a change in the presence of C. difficile in the intestinal flora (e.g., an increase in the total amount of C. difficile relative to one or more other bacteria, etc.), which gives rise or may give rise to adverse effect(s) and/or an increase in the level of toxins A and/or B in the intestine or other organs and tissues comprising the gastrointestinal tract. Typically, CDAD results from the acquisition and proliferation of C. difficile in the gut. In vivo, toxins A and B demonstrate different pathological profiles with potential synergy in causing disease. In rabbits and mice, for example, toxin A is an enterotoxin that induces diarrhea, while toxin B does not elicit a fluid response in this species. However, toxin B is more potently cytotoxic in vitro. Toxin A-negative, toxin B-positive (A− B+) strains of C. difficile have been increasingly reported. A−/B+ strains fail to produce toxin A due to deletion of the repetitive domain of the tcdA gene, yet are still capable of causing clinical disease. In contrast, there are to date no reports of toxin A-positive, toxin B-negative (A+/B−) strains in humans.

C. difficile infection commonly manifests as mild-to-moderate diarrhea, occasionally with abdominal cramping. Pseudomembranes, which are adherent yellowish-white plaques on the intestinal mucosa, are occasionally observed. In rare cases, patients with C. difficile infection can present with an acute abdomen and fulminant life-threatening colitis, which results from a disruption of the normal bacterial flora of the colon, colonization with C. difficile and release of toxins that cause mucosal inflammation and damage. Antibiotic therapy is the key factor that alters the colonic flora. While normal gut flora resists colonization and overgrowth with C. difficile, antibiotic use, which suppresses the normal flora, allows C. difficile bacteria to proliferate. C. difficile is present in 2-3% of healthy adults and in as many as 70% of healthy infants. In one of its aspects, the mAbs of the present invention are utilized for the treatment of subjects who are asymptomatic, but who are susceptible to, or at risk of, contracting C. difficile infection and becoming afflicted with its associated diseases. Such subjects may be hospitalized or may be outside of a hospital setting.

The chief risk factor for C. difficile-associated disease is prior exposure to antibiotics. The most common antibiotics implicated in C. difficile colitis include cephalosporins (especially second and third generation), ampicillin/amoxicillin and clindamycin. Less commonly implicated antibiotics are the macrolides (i.e., erythromycin, clarithromycin, azithromycin) and other penicillins. Compounds or other agents which are occasionally reported to cause the disease include aminoglycosides, fluoroquinolones, trimethoprim-sulfamethoxazole, metronidazole, chloramphenicol, tetracycline, imipenem, and meropenem. Even brief exposure to any single antibiotic can cause C. difficile colitis, particularly if normal intestinal flora are adversely affected or killed. A prolonged antibiotic course, or the use of two or more antibiotics, increases the risk of disease. Antibiotics traditionally used to treat C. difficile colitis have been shown to cause disease. Other risk factors associated with infection by C. difficile include advanced age (>65 years); weakened immune system; recent hospitalization (particularly sharing a hospital room with an infected patient, intensive care unit stays and prolonged hospital stays); living in a nursing home, hospice, or other longterm care facility; abdominal surgery; chronic colon disease, (e.g., inflammatory bowel disease (IBD) or colorectal cancer); taking prescription or over the counter antacids which may reduce stomach acid and allow C. difficile to pass more easily into the intestine; and a previous C. difficile infection. More factors associated with C. difficile disease include antineoplastic agents, principally methotrexate, hemolytic-uremic syndrome, malignancies, intestinal ischemia, renal failure, necrotizing enterocolitis, Hirschsprung disease, IBD and nonsurgical gastrointestinal procedures, including nasogastric tubes. The subjects that can be administered the therapies provided herein include any of the subjects described that are at risk for C. difficile infection.

While most patients with C. difficile colitis recover without specific therapy, symptoms may be prolonged and debilitating. C. difficile-associated diarrhea can be a serious condition with a mortality rate of up to 25% in elderly patients who are frail. Reports that focus on more seriously ill patients indicate mortality rates of 10-30%. C. difficile infection is more common in elderly people, and old age may promote susceptibility to colonization and disease. While infants and young children frequently harbor C. difficile and its toxins, clinical infection is uncommon. Cross-infection by C. difficile is common in neonatal units, but neonates do not seem to develop C. difficile-associated diarrhea.

### Therapeutic Methods

The disclosure provides methods of treating C. difficile infection, C. difficile-associated disease, and symptoms thereof, comprising the use of one or more isolated antibodies having enhanced half-life, or antigen-binding fragments thereof, which inhibit, block, or prevent C. difficile toxin A and/or toxin B toxicity or activity. C. difficile pathology is driven by two secreted toxins, A and B, which mediate the colitis, diarrhea and massive inflammatory response characteristic of this disease. Toxins A and B are the major virulence determinants of C. difficile, and toxin-negative strains are nonpathogenic. Toxins A and B are transcribed from a pathogenicity locus that includes the toxin genes, tcdA (toxin A) and tcdB (toxin B), and three regulatory genes, one of which (tcdC) encodes a putative negative regulator of toxin transcription. TcdC protein appears to inhibit toxin transcription during the early, exponential-growth phase of the bacterial life cycle. For toxin B, an autocatalytic cleavage site between leucine543 and glycine544 has been described. Cleavage results from activation of an aspartyl protease domain by host cytosolic inositol phosphate, and releases the active glucosyltransferase domain.

Toxin-neutralizing antibodies have previously demonstrated clinical benefit in reducing the recurrence of CDI. PA50YTE/PA41YTE COMBINATION is an equimolar mixture of two fully human monoclonal antibodies having enhanced half-life which bind to and neutralize the cytotoxicity of toxins A and B. In the hamster infection model, PA50YTE/PA41YTE COMBINATION was more effective than vancomycin in treating lethal C. difficile infections. Compared to the antitoxin antibodies currently in clinical trials, PA50YTE/PA41YTE COMBINATION demonstrated greater toxin neutralizing potency in vitro and neutralized toxins from a broader range of clinical isolates. Importantly, in the hamster infection model, PA50YTE/PA41YTE COMBINATION provided superior protection when compared to existing antitoxin monoclonal antibodies. In addition, the monoclonal antibodies that comprise PA50YTE/PA41YTE COMBINATION are engineered with extended half-life technology providing a 3-fold expanded window of toxin neutralization compared to standard IgG, providing months of prophylaxis against infection recurrence.

Treatment of C. difficile infections with PA50YTE/PA41YTE COMBINATION as monotherapy, or in combination with a brief course of antibiotics, should provide rapid abatement of clinical signs and symptoms. The elimination or minimization of antibiotic exposure made possible by PA50YTE/PA41YTE COMBINATION treatment should allow patients to re-establish their protective microbiome sooner than would be possible with a full course of standard antibiotic therapy. Treatment with anti-toxin A and anti-toxin B antibodies having enhanced half-life can allow for the restoration of normal gut flora in a subject infected with C. difficile. Such antibodies can resolve disease in patients undergoing treatment. Treatment with anti-toxin A and anti-toxin B antibodies having enhanced half-life can also demonstrate beneficial in vivo pharmacokinetics. Treatment with anti-toxin A and anti-toxin B antibodies having enhanced half-life can also provide prolonged or long lasting therapy for a subject who has been infected with C. difficile. As used herein, “long lasting” refers to therapy that results in an absence of C. difficile infection or C. difficile-associated disease one month or more after cessation of treatment. Preferably, the therapy results in an absence of C. difficile infection or C. difficile-associated disease for two or more months. In some embodiments, therapy with mAbs of the invention results in treating or depressing active C. difficile infection and in reducing or diminishing the robustness of infection. In other embodiments, therapy provided by the invention results in an absence of C. difficile infection or C. difficile-associated disease in a subject for 1, 2, 3, 4, 5, or 6 months. In other embodiments, therapy provided by the invention results in an absence of C. difficile infection or C. difficile-associated disease in a subject for longer than 6 months. Treatment with anti-toxin A and anti-toxin B antibodies having enhanced half-life can prevent recurrence of C. difficile infection and/or C. difficile-associated disease.

As another example, treatment with anti-toxin A and anti-toxin B antibodies having enhanced half-life can effect a cure or survival rate of at least 50%, 55%, 60%, 65%, 70%, 75%, 80%, 85%, 90%, 95%, 96%, 97%, 98%, 99%, or even 100%. As another example, the antibodies can effect a cure or survival rate of 100%. In one embodiment, one or more anti-toxin A antibodies, when administered to a subject, together with one or more anti-toxin B antibodies, effect a cure or survival rate of 50%, 55%, 60%, 65%, 70%, 75%, 80%, 85%, 90%, 95%, 97%, 99%, or 100%. As used herein, “cure rate” refers to the percentage of subjects that a clinician would determine to no longer have the infection or disease out of a population of subjects with the infection or disease administered one or more antibodies, or one or more therapeutic methods thereof, of the invention. “Survival rate”, as used herein, refers to the percentage of subjects that survive for a desired period of time out of a population of subjects administered one or more antibodies, or one or more therapeutic methods thereof, of the invention.

The long serum half-life of PA50YTE/PA41YTE COMBINATION also provides a continuous window of toxin neutralization further minimizing the recurrence of CDI. In summary, PA50YTE/PA41YTE COMBINATION is an example of a precision medicine that effectively treats a difficult bacterial infection without the collateral damage to the beneficial microbiome associated with traditional antibiotic therapy.

### PA50YTE/PA41YTE COMBINATION and Vancomycin Treatment Regimen

As reported in detail below, PA50YTE/PA41YTE COMBINATION is at least as effective as vancomycin in treating C. difficile infections. PA50YTE/PA41YTE COMBINATION acts by competitively inhibiting toxin binding to the intestinal wall, thereby rendering the wall less susceptible to C. difficile infection. In contrast, vancomycin is a bactericidal agent. In particular embodiments, vancomycin and PA50YTE/PA41YTE COMBINATION may be administered concurrently. Such combined therapeutic strategy would likely require a lower dose or reduced frequency of administration of vancomycin than conventional vancomycin therapy, thereby reducing adverse side effects, enhancing microbiome restoration, reducing microbiome dysbiosis, and/or reducing the risk of re-infection.

Conventional vancomycin dosage and administration are described and known in the art (see e.g., Rybak et al., Am J Health Syst Pharm. 2009; 66(1):82-98; American Society of Health-System Pharmacists, the Infectious Diseases Society of America, and the Society of Infectious Diseases Pharmacists). Vancomycin dosages are calculated on actual body weight (ABW). However, for obese patients, initial dosing is based on ABW and then adjusted based on serum vancomycin concentrations to achieve therapeutic levels. Vancomycin dosages of 15-20 mg/kg (based on ABW) given every 8-12 hours achieve target serum concentrations of MIC≦1 mg/L in most patients with normal renal function (e.g., 1 g every 12 hours). In one embodiment, a maintenance dose (about 15-20 mg/kg of actual body weight, rounded to the nearest 250 mg) is administered at the dosing interval recommended for a patient's creatinine clearance levels (CrCL) (see Table 2). Maximum initial dose is about 1750 mg about every 12 hours until serum concentration monitoring indicates the need for higher dosing. Exemplary vancomycin maintenance doses and infusion rates are provided at Table 1.

TABLE 1
Vancomycin Maintenance Doses and Infusion Rates

| VANCOMYCIN | | INFUSION RATE |
|---|---|---|
| MAINTENANCE DOSES | | BASED ON DOSE |
| Total body wt (kg) | Dose (mg) | (approx. ≤ 15 mg/min) |
| ≥111 | 1750 | 120 minutes |
| 90-110 | 1500 | 90 minutes |
| 75-89 | 1250 | 75 minutes |
| 60-74 | 1000 | 60 minutes |
| 50-59 | 750 | 60 minutes |
| 30-49 | 500 | 50 minutes |

In order to achieve rapid attainment of this target concentration for seriously ill patients, a loading dose of 25-30 mg/kg (based on ABW) can be used. In one embodiment, a one-time loading dose of about 25-30 mg/kg of actual body weight (rounded to the nearest 250 mg) at a rate of about 500 mg/hour (but no more than about 1 g/hr) may be considered for seriously ill patients (e.g., sepsis, fever and neutropenia, suspected/proven MRSA bacteremia) with CrCL>30 mL/min to rapidly attain therapeutic concentrations. Exemplary vancomycin loading doses and infusion rates are provided at Table 2.

TABLE 2
Vancomycin Loading Doses and Infusion Rates

| VANCOMYCIN | | INFUSION RATE |
|---|---|---|
| LOADING DOSES | | BASED ON DOSE |
| Total body wt (kg) | Dose (mg) | (approx. ≤ 15 mg/min) |
| ≥90 | 3000 | 360 minutes |
| 75-89 | 2500 | 300 minutes |
| 60-74 | 2000 | 240 minutes |
| 50-59 | 1500 | 180 minutes |
| 30-49 | 1000 | 120 minutes |

Individual pharmacokinetic adjustments and verification of serum target achievement are recommended.

Vancomycin should be administered intravenously over an infusion period of at least 1 hour to minimize infusion related adverse effects. Vancomycin may be administered by intermittent dosing or continuous infusion. When individual doses exceed 1 g (i.e., 1.5 and 2 g), the infusion period should be extended to 1.5-2 hours. Vancomycin dosing intervals are based in part on a patient's creatinine clearance levels (CrCL). For example, vancomycin dosing intervals based on estimated CrCL are provided at Table 3.

TABLE 3
Vancomycin Dosing Interval Based on Estimated Creatinine Clearance Level
(CrCL).
VANCOMYCIN DOSING INTERVAL BASED ON ESTIMATED CrCL

| CrCL (mL/min) | Dosing interval |
|---|---|
| ≥100 | Q8-12 h |
| | (Consider Q8 h dosing if <50 years old with severe infection and |
| | normal renal function) |
| 50-99 | Q12 h |
| 30-49 | Q24 h |
| <30 * | Initial loading dose of 15-20 mg/kg. Redose with 15 mg/kg when serum |
| Hemodialysis | level ≤ 15 mg/L or when ≤ 20 mg/L in severe infections where |
| Peritoneal dialysis | penetration may be compromised (e.g., meningitis, pneumonia) |
| Continuous renal replacement | Q24-48 h |
| therapy (CRRT) | (Maintains trough 10-15 mg/L or 15-20 mg/L in severe infections where |
| | penetration may be compromised (e.g., meningitis, pneumonia)) |

For the treatment of pseudomembranous colitis, vancomycin may be administered orally to reach the site of infection in the colon. For treatment of C. difficile infection in adults, a conventional regimen is vancomycin administered orally at about 125 mg about every 6 hr for 10 days. In children, a conventional regimen is vancomycin administered orally at about 40 mg/kg/day about every 6-8 hours for 7-10 days; not to exceed 2 g/day. Following oral administration, the fecal concentration of vancomycin may be about 500 μg/ml (Edlund et al., Clinical Infectious Diseases, 1997; 25 (3): 729-32) compared to MIC≦2 μg/ml for sensitive strains of C. difficile (Pelaez et al., Antimicrob Agents Chemother, 2002; 46 (6): 1647-1650).

Trough serum vancomycin concentrations are the most accurate and practical method for monitoring vancomycin effectiveness. Trough concentrations should be obtained just before the next dose at steady state conditions. Steady-state achievement is variable and dependent on multiple factors. Trough samples should be obtained just before the fourth dose in patients with normal renal function to ensure that target concentrations are attained. Based on the potential to improve penetration, increase the probability of optimal target serum vancomycin concentrations, and improve clinical outcomes for infections, total trough serum vancomycin concentrations of 15-20 mg/L are recommended. Trough serum vancomycin concentrations in that range should achieve an AUC (area under the concentration-versus-time curve)/MIC (minimum inhibitory concentration) of ≧400 in most patients if the MIC is ≦1 mg/L. In order to achieve rapid attainment of this target concentration for seriously ill patients, a loading dose of 25-30 mg/kg (based on ABW) can be considered.

An AUC/MIC ratio of ≧400 has been advocated as a target to achieve clinical effectiveness with vancomycin. Animal studies and limited human data appear to demonstrate that vancomycin is not concentration dependent and that the AUC/MIC is a predictive pharmacokinetic parameter for vancomycin. Based on evidence suggesting that exposure to trough serum vancomycin concentrations of <10 mg/L can produce strains with resistance, it is recommended that trough serum vancomycin concentrations always be maintained above 10 mg/L to avoid development of resistance. A targeted AUC/MIC of ≧400 is not achievable with conventional dosing methods if the vancomycin MIC is ≧2 mg/L in a patient with normal renal function (i.e., CrCL of 70-100 mL/min). Therefore, alternative therapies should be considered.

Vancomycin has long been considered a nephrotoxic and ototoxic agent. A patient should be identified as having experienced vancomycin-induced nephrotoxicity if multiple (at least two or three consecutive) high serum creatinine concentrations (increase of 0.5 mg/dL or ≧50% increase from baseline, whichever is greater) are documented after several days of vancomycin therapy in the absence of an alternative explanation.

Monitoring of trough serum vancomycin concentrations to reduce nephrotoxicity is best suited to patients receiving aggressive dosing targeted to produce sustained trough drug concentrations of 15-20 mg/L or who are at high risk of toxicity, such as patients receiving concurrent nephrotoxins. When this target range is desired, obtaining once-weekly trough concentrations in hemodynamically stable patients is recommended. Patients receiving prolonged courses of vancomycin should have at least one steady-state trough concentration obtained (just before the fourth dose). Monitoring is also recommended for patients with unstable renal function (either deteriorating or significantly improving) and those receiving prolonged courses of therapy (over three to five days). Frequent (in some instances daily) trough concentration monitoring is advisable to prevent toxicity in hemodynamically unstable patients. The exact frequency of monitoring is often a matter of clinical judgment.

Anti-C. difficile Toxin a and Toxin B Antibodies

The therapeutic methods described herein comprise the use of one or more isolated antibodies having enhanced half-life, including antigen-binding fragments and modified derivatives thereof, which inhibit, block, or prevent C. difficile toxin A and/or toxin B toxicity or activity. Exemplary anti-tcdA (e.g., PA-50, PA-39, and PA-38) and anti-tcdB antibodies (e.g., PA-41) are described in US20130202618/U.S. Pat. No. 8,986,697, each of which is incorporated herein by reference in their entireties. Exemplary antibodies may also comprise one or more of the VH, VL, heavy chain, and light chain sequences at SEQ ID NOs: 7-22.

In one aspect, the invention provides methods of treatment comprising the use of an isolated antibody, or antigen-binding fragment thereof, which inhibits, blocks, or prevents toxin A internalization and cytocellular toxicity. In certain embodiments, the antibody is a monoclonal antibody. In particular embodiments, the antibody is a humanized or chimeric antibody. In specific embodiments, the antibody is PA-50 (ATCC Accession No. PTA-964) or humanized PA-50. In other embodiments, the antibody is PA-39 (ATCC Accession No. PTA-9692) or humanized PA-39. In various embodiments, the antibody binds toxin A outside of the receptor binding domain of toxin A of C. difficile.

In another aspect, the methods comprise the use of isolated antibody, or antigen-binding fragment thereof, which inhibits, blocks, or prevents C. difficile toxin B toxicity by binding to an epitopic site in the N-terminal enzymatic region of toxin B. In certain embodiments, the antibody is a monoclonal antibody. In particular embodiments, the antibody is a humanized or chimeric antibody. In specific embodiments, the antibody is PA-41 (ATCC Accession No. PTA-9693) or a humanized form of PA-41. In various embodiments, the antibody binds to the N-terminal enzymatic region of toxin B of C. difficile.

The antibodies of the invention exhibit a number of beneficial characteristics. For example, the anti-toxin A antibodies neutralize or inhibit the toxicity of toxin A both in vitro and in vivo. In in vitro neutralization studies, humanized PA-39 and humanized PA-41 demonstrated neutralization potencies (i.e., EC₅₀ values; US20130202618/U.S. Pat. No. 8,986,697) higher than those compared with values for neutralization by other human anti-toxin A and anti-toxin B monoclonal antibodies that have been reported (WO/2006/121422; US2005/0287150; Babcock et al., Infect. Immun., 2006).

In various embodiments, the invention provides treatment with antibodies having enhanced half-lives. Anti-C. difficile toxin antibodies (e.g., PA-39, PA-41, PA-50) can be linked to another functional molecule, e.g., another peptide or protein (e.g., albumin). For example, the antibodies can be linked by chemical cross-linking or by recombinant methods. The antibodies may also be linked to one of a variety of nonproteinaceous polymers, e.g., polyethylene glycol, polypropylene glycol, or polyoxyalkylenes, in the manner set forth in U.S. Pat. No. 4,640,835; 4,496,689; 4,301,144; 4,670,417; 4,791,192; or 4,179,337. The antibodies can be chemically modified by covalent conjugation to a polymer, for example, to increase their circulating half-life. Exemplary polymers and methods to attach them are also shown in U.S. Pat. Nos. 4,766,106; 4,179,337; 4,495,285, and 4,609,546.

In certain embodiments, the Fc region of the antibody comprises at least one non-naturally occurring amino acid at one or more positions chosen from 252, 254, and 256. In various embodiment, the non-naturally occurring amino acids are selected from the group chosen from 252Y, 254T and 256E (referred to as the “YTE modification”), as described in Dall'Acqua et al., J. Biol. Chem., 281, 23514-23524 (2006), and in U.S. Pat. No. 7,083,784/US20030190311, each of which is incorporated herein by reference in their entireties. Antibodies having the YTE modification have enhanced half-lives compared to the unmodified antibodies (e.g., the parent antibody). In one embodiment, PA-50-YTE is a fully human monoclonal antibody having enhanced half-life which binds to and neutralizes the cytotoxicity of toxin A. In one embodiment, PA-41-YTE is a fully human monoclonal antibody having enhanced half-life which binds to and neutralizes the cytotoxicity of toxin B. In one aspect, the invention features a composition comprising an equimolar mixture of the anti-toxin A antibody PA-50-YTE and anti-toxin B antibody PA-41-YTE termed PA50YTE/PA41YTE COMBINATION (also termed PA50YTE/PA40YTE COMBINATION in priority application U.S. 62/147,908 filed on 15 Apr. 2015).

In one embodiment, an anti-toxin A antibody neutralizes or inhibits the in vivo toxicity of C. difficile toxin A at an effective dose. In another embodiment, the anti-toxin B antibodies neutralize or inhibit the in vivo toxicity of toxin B. In an embodiment, an effective dose of one or more anti-toxin A antibodies is provided to a C. difficile-infected subject. In an embodiment, an effective dose of one or more anti-toxin A antibodies of the invention is provided in combination with an effective dose of one or more anti-toxin B antibodies of the invention to a C. difficile-infected subject. In an embodiment, an anti-toxin A antibody of the invention in a 1:1 combination with an anti-toxin B antibody of the invention is provided as an effective dose to a C. difficile-infected subject. In an embodiment, an effective dose of an anti-toxin A antibody and an anti-toxin B antibody of the invention may be, for example, a 1:1, 1:1, 2:1, 3:1, 4:1, etc., combination of the antibodies provided to a C. difficile-infected subject. In an embodiment, the antibodies are humanized. In an embodiment, the antibodies are included in a composition.

Illustratively, an effective dose of the anti-toxin A and/or anti-toxin B antibodies may range from 0.1 μg to 1000 milligrams (mg). The anti-toxin A antibodies and anti-toxin B antibodies or antigen-binding fragments thereof may be administered to a subject in an amount of, for example, 0.1 mg/kg-150 mg/kg; in an amount of 0.5 mg/kg-75 mg/kg; in an amount of 1 mg/kg-100 mg/kg; in an amount of 1 mg/kg-50 mg/kg; in an amount of 2 mg/kg-40 mg/kg; in an amount of 2 mg/kg-50 mg/kg; in an amount of 5 mg/kg-50 mg/kg; in an amount of 5 mg/kg-25 mg/kg; in an amount of 10 mg/kg-40 mg/kg; in an amount of 10 mg/kg-50 mg/kg; in an amount of 10 mg/kg-25 mg/kg; or in an amount of 15 mg/kg-50 mg/kg. In an embodiment, the aforementioned amounts may comprise the varying ratios of anti-toxin A antibody and anti-toxin B antibody provided in combination.

In some embodiments, the dose or amount of the one or more anti-toxin A or anti-toxin B antibodies may range for example from 0.2 μg-250 μg, or from 2 μg-50 μg, or from 5 μg-50 μg, e.g., based on in vivo mouse studies. In some embodiments, the dose or amount of one or more anti-toxin A or anti-toxin B antibodies, and in particular a combination of an anti-toxin A antibody and an anti-toxin B antibody, may range for example from 2 mg/kg-40 mg/kg, 2 mg/kg-50 mg/kg, 5 mg/kg-40 mg/kg, 5 mg/kg-50 mg/kg, 10 mg/kg-40 mg/kg, or 10 mg/kg-50 mg/kg, e.g., based on in vivo hamster studies.

Antibodies provided herein include monoclonal antibodies produced by hybridomas that were deposited and given the following Patent Deposit Designations: PTA-9692 (for PA-39), PTA-9693 (for PA-41), PTA-9694 (for PA-50), and PTA-9888 (for PA-38). These hybridomas were deposited pursuant to, and in satisfaction of, the requirements of the Budapest Treaty on the International Recognition of the Deposit of Microorganisms for the Purposes of Patent Procedure with the American Type Culture Collection (“ATCC”), P.O. Box 1549, Manassas, Va. 20108 USA, as an International Depository Authority, on Jan. 6, 2009 (for PTA-9692, PTA-9693, PTA-9694) and on Mar. 24, 2009 (for PTA-9888) and given the aforementioned Patent Deposit Designations. As used herein, both the deposited hybridomas and the monoclonal antibodies produced by the hybridomas may be referred to by the same ATCC Deposit Designations or to the numbers found within the ATCC Deposit Designations. For example, PTA-9888 or 9888 may be used to refer to the deposited hybridoma or to the monoclonal antibody produced by the hybridoma. Accordingly, the names of the monoclonal antibodies described herein may be used interchangeably with the names of the hybridomas that produce them. It will be clear to one of skill in the art when the name is intended to refer to the antibody or to the hybridoma that produces the antibody. The antigen-binding fragments provided herein include the antigen-binding fragments of the aforementioned deposited antibodies.

### Methods of Antibody Production

Antibodies can be made, for example, using traditional hybridoma techniques (Kohler and Milstein (1975) Nature, 256: 495-499), recombinant DNA methods (U.S. Pat. No. 4,816,567), or phage display performed with antibody, libraries (Clackson et al. (1991) Nature, 352: 624-628; Marks et al. (1991) J. Mol. Biol., 222: 581-597). For other antibody production techniques, see also Antibodies: A Laboratory Manual, eds. Harlow et al., Cold Spring Harbor Laboratory, 1988. The invention is not limited to any particular source, species of origin, or method of production.

Intact antibodies, also known as immunoglobulins, are typically tetrameric glycosylated proteins composed of two light (L) chains of approximately 25 kDa each and two heavy (H) chains of approximately 50 kDa each. Two types of light chain, designated as the λ chain and the κ chain, are found in antibodies. Depending on the amino acid sequence of the constant domain of heavy chains, immunoglobulins can be assigned to five major classes: A, D, E, G, and M, and several of these may be further divided into subclasses (isotypes), e.g., IgG₁, IgG₂, IgG₃, IgG₄, IgA₁, and IgA₂.

The subunit structures and three-dimensional configurations of different classes of immunoglobulins are well known in the art. For a review of antibody structure, see Harlow et al., supra. Briefly, each light chain is composed of an N-terminal variable domain (V_{L}) and a constant domain (C_{L}). Each heavy chain is composed of an N-terminal variable domain (V_{H}), three or four constant domains (C_{H}), and a hinge region. The C_{H} domain most proximal to V_{H} is designated as C_{H}1. The V_{H} and V_{L} domains consist of four regions of relatively conserved sequence called framework regions (FR1, FR2, FR3, and FR4), which form a scaffold for three regions of hypervariable sequence called complementarity determining regions (CDRs). The CDRs contain most of the residues responsible for specific interactions with the antigen. The three CDRs are referred to as CDR1, CDR2, and CDR3. CDR constituents on the heavy chain are referred to as H1, H2, and H3, while CDR constituents on the light chain are referred to as L1, L2, and L3, accordingly. CDR3 and, particularly H3, are the greatest source of molecular diversity within the antigen-binding domain. H3, for example, can be as short as two amino acid residues or greater than 26. In particular embodiments, a heavy chain CDR3 (H3) comprises between about 4 amino acids (see, for example, Ab No. 2) and 22 amino acids (see, for example, Ab Nos. 20 and 34).

The Fab fragment (Fragment antigen-binding) consists of the V_{H}-C_{H}1 and V_{L}-C_{L} domains covalently linked by a disulfide bond between the constant regions. To overcome the tendency of non-covalently linked V_{H} and V_{L} domains in the Fv to dissociate when co-expressed in a host cell, a so-called single chain (sc) Fv fragment (scFv) can be constructed. In a scFv, a flexible and adequately long polypeptide links either the C-terminus of the V_{H} to the N-terminus of the V_{L} or the C-terminus of the V_{L} to the N-terminus of the V_{H}. Most commonly, a 15-residue (Gly₄Ser)₃ peptide is used as a linker, but other linkers are also known in the art.

Antibody diversity is a result of combinatorial assembly of multiple germline genes encoding variable regions and a variety of somatic events. The somatic events include recombination of variable gene segments with diversity (D) and joining (J) gene segments to make a complete V_{H} region and the recombination of variable and joining gene segments to make a complete V_{L} region. The recombination process itself is imprecise, resulting in the loss or addition of amino acids at the V(D)J junctions. These mechanisms of diversity occur in the developing B cell prior to antigen exposure. After antigenic stimulation, the expressed antibody genes in B cells undergo somatic mutation.

Based on the estimated number of germline gene segments, the random recombination of these segments, and random V_{H}-V_{L} pairing, up to 1.6×10⁷ different antibodies could be produced (Fundamental Immunology, 3rd ed., ed. Paul, Raven Press, New York, N.Y., 1993). When other processes that contribute to antibody diversity (such as somatic mutation) are taken into account, it is thought that upwards of 1×10¹⁰ different antibodies could be potentially generated (Immunoglobulin Genes, 2^{nd} ed., eds. Jonio et al., Academic Press, San Diego, Calif., 1995). Because of the many processes involved in antibody diversity, it is highly unlikely that independently generated antibodies will have identical or even substantially similar amino acid sequences in the CDRs.

The structure for carrying a CDR will generally be an antibody heavy or light chain or a portion thereof, in which the CDR is located at a location corresponding to the CDR of naturally occurring V_{H} and V_{L}. The structures and locations of immunoglobulin variable domains may be determined, for example, as described in Kabat et al., Sequences of Proteins of Immunological Interest, No. 91-3242, National Institutes of Health Publications, Bethesda, Md., 1991.

Anti-C. difficile toxin A and toxin B antibodies may optionally comprise antibody constant regions or parts thereof. For example, a V_{L} domain may have attached, at its C terminus, antibody light chain constant domains including human Cκ or Cλ chains. Similarly, a specific antigen-binding domain based on a V_{H} domain may have attached all or part of an immunoglobulin heavy chain derived from any antibody isotope, e.g., IgG, IgA, IgE, and IgM and any of the isotope sub-classes, which include but are not limited to, IgG₁ and IgG₄. The DNA and amino acid sequences for the C-terminal fragment of are well known in the art (see, e.g., Kabat et al., Sequences of Proteins of Immunological Interest, No. 91-3242, National Institutes of Health Publications, Bethesda, Md., 1991).

Certain embodiments comprise a V_{H} and/or V_{L} domain of an Fv fragment from a C. difficile toxin A or toxin B antibody. Further embodiments comprise at least one CDR of any of these V_{H} and V_{L} domains. In certain embodiments, the V_{H} and/or V_{L} domains may be germlined, i.e., the framework regions (FRs) of these domains are mutated using conventional molecular biology techniques to match those produced by the germline cells. In other embodiments, the framework sequences remain diverged from the consensus germline sequences.

One of ordinary skill in the art will recognize that the antibodies of this invention may be used to inhibit proteins that differ somewhat from toxin A or toxin B. The antibodies are expected to retain the specificity of binding so long as the target protein comprises a sequence which is at least about 60%, 70%, 80%, 90%, 95%, or more identical to any sequence of at least 100, 80, 60, 40, or 20 of contiguous amino acids of toxin A or toxin B. The percent identity is determined by standard alignment algorithms such as, for example, Basic Local Alignment Tool (BLAST) described in Altshul et al. (1990) J. Mol. Biol., 215: 403-410, the algorithm of Needleman et al. (1970) J. Mol. Biol., 48: 444-453, or the algorithm of Meyers et al. (1988) Comput. Appl. Biosci., 4: 11-17.

In addition to the sequence homology analyses, epitope mapping (see, e.g., Epitope Mapping Protocols, ed. Morris, Humana Press, 1996) and secondary and tertiary structure analyses can be carried out to identify specific 3D structures assumed by the disclosed antibodies and their complexes with antigens. Such methods include, but are not limited to, X-ray crystallography (Engstom (1974) Biochem. Exp. Biol., 11:7-13) and computer modeling of virtual representations of the presently disclosed antibodies (Fletterick et al. (1986) Computer Graphics and Molecular Modeling, in Current Communications in Molecular Biology, Cold Spring Harbor Laboratory, Cold Spring Harbor, N.Y.).

### Kits

The invention provides kits for treating a C. difficile infection or symptoms thereof. In one embodiment, the kit includes a therapeutic composition containing an effective amount of one or more of an anti-toxin A antibody and/or anti-toxin B antibody having enhanced half-life in unit dosage form.

In some embodiments, the kit comprises a sterile container which contains a therapeutic or prophylactic biological composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired an antibody of the invention is provided together with instructions for administering the antibody or agent to a subject having or at risk of developing C. difficile infection, C. difficile associated disease, or symptoms thereof. The instructions will generally include information about the use of the antibodies for the treatment or prevention of such indications. In other embodiments, the instructions include at least one of the following: description of the therapeutic agent; dosage schedule and administration for treatment or prevention of a C. difficile infection or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow. The following examples are put forth so as to provide those of ordinary skill in the art with a complete disclosure and description of how to make and use the anti-P2X4 antibodies in assay, screening, and therapeutic methods of the invention, and are not intended to limit the scope of what the inventors regard as their invention.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Treatment with a Combination of Anti-Toxin a and Anti-Toxin B Monoclonal Antibodies Increased Survival and Protected Against Toxicity in a Model of C. difficile Infection

The hamster model of C. difficile infection reproduces key aspects of C. difficile-Associated Diarrhea (CDAD) disease in humans. Upon treatment with antibiotics, normal colonic flora is eradicated and the hamsters become readily susceptible to infection by C. difficile. Infection results in severe diarrhea, pseudomembranous colitis and death. The hamster CDAD model was utilized to evaluate the potential efficacy of monoclonal anti-toxin A and anti-toxin B antibodies to prevent disease and death associated with challenge of animals from live C. difficile bacteria.

Hamsters were challenged with C. difficile spores by oral administration at day 0 and pretreated with a single dose of clindamycin (10 mg/kg) at day 1 to disrupt the normal colonic flora. Animals were placed in a control group receiving no treatment and groups receiving either vancomycin (on days 2, 3, 4, 5, and 6) or a combination of toxin A and toxin B antibodies PA-50-YTE (40 mg/kg) and PA-41-YTE (40 mg/kg), also termed MEDI095, on day 2. Animals were monitored daily for health status and survival.

All hamsters in the infection control group that did not receive treatment were dead by day 3 of the study. In the vancomycin-treated group, treatment extended survival beyond 3 days in a majority of the animals. However, after discontinuation of therapy most of the animals (˜80%) were dead by day 21 at the conclusion of the study. In contrast, all animals receiving a combination of antibodies PA-50-YTE and PA-41-YTE (i.e., MEDI095) showed 100% survival up to 21 days post-challenge. Accordingly, treatment with PA50YTE/PA41YTE COMBINATION provided a superior and sustained post infection protective benefit relative to antibiotic treatment.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

SEQUENCE LISTING

```
SEQ ID NO: 1 PA50-YTE Heavy Chain
qvqlvqsgaevkkpgasvkvsckasgytftdynmdwvrqapgqrlewmgdinpkydiighnpkfmgrvti
trdtsastaymelsslrsedtavyycarsdrgwyfdvwgqgtlvtvssastkgpsvfplapsskstsggt
aalgclvkdyfpepvtvswnsgaltsgvhtfpavlqssglyslssvvtvpssslgtqtyicnvnhkpsnt
kvdkrvepkscdkthtcppcpapellggpsvflfppkpkdtlyitrepevtcvvvdvshedpevkfnwyv
dgvevhnaktkpreeqynstyrvvsvltvlhqdwlngkeykckvsnkalpapiektiskakgqprepqvy
tlppsreemtknqvsltclvkgfypsdiavewesngqpennykttppvldsdgsfflyskltvdksrwqq
gnvfscsvmhealhnhytqkslslspgk
SEQ ID NO: 2 PA50-YTE Light Chain
eivltqspatlslspgeratlscrasssvnymnwyqqkpgqaprpliyatsnlasgiparfsgsgsgtdf
tltisslepedfavyycqqwssrtfgggtkleikrtvaapsvfifppsdeqlksgtasvvcllnnfypre
akvqwkvdnalqsgnsqesvteqdskdstyslsstltlskadyekhkvyacevthqglsspvtksfnrge
c
SEQ ID NO: 3 PA41-YTE Heavy Chain
qvqlvqsgaevkkpgasvkvsckasgypftnyfmhwvrqapgqrlewigrinpyngatsyslnfrdkati
tldksastaymelsslrsedtavyycarstitsplldfwgqgtlvtvssastkgpsvfplapsskstsgg
taalgclvkdyfpepvtvswnsgaltsgvhtfpavlqssglyslssvvtvpssslgtqtyicnvnhkpsn
tkvdkrvepkscdkthtcppcpapellggpsvflfppkpkdtlyitrepevtcvvvdvshedpevkfnwy
vdgvevhnaktkpreeqynstyrvvsvltvlhqdwlngkeykckvsnkalpapiektiskakgqprepqv
ytlppsreemtknqvsltclvkgfypsdiavewesngqpennykttppvldsdgsfflyskltvdksrwq
qgnvfscsvmhealhnhytqkslslspgk
SEQ ID NO: 4 PA41-YTE Light Chain
eivltqspatlslspgeratlscrasqsvgtsihwyqqkpgqaprllikfasesisgiparfsgsgsgtd
ftltisslepedfavyycqqsnkwpftfgqgtkleikrtvaapsvfifppsdeqlksgtasvvcllnnfy
preakvqwkvdnalqsgnsqesvteqdskdstyslsstltlskadyekhkvyacevthqglsspvtksfn
rgec
SEQ ID NO: 5 Clostridium difficile toxin A (TcdA)
      1 msliskeeli klaysirpre neyktiltnl deynklttnn nenkylqlkk lnesidvfmn
    61 kyktssrnra lsnlkkdilk eviliknsnt spveknlhfv wiggevsdia leyikqwadi
  121 naeyniklwy dseaflvntl kkaivesstt ealqlleeei qnpqfdnmkf ykkrmefiyd
  181 rqkrfinyyk sqinkptvpt iddiikshlv seynrdetvl esyrtnslrk insnhgidir
  241 anslfteqel lniysqelln rgnlaaasdi vrllalknfg gvyldvdmlp gihsdlfkti
  301 srpssigldr wemikleaim kykkyinnyt senfdkldqq lkdnfkliie sksekseifs
  361 klenlnvsdl eikiafalgs vinqaliskq gsyltnlvie qvknryqfln qhlnpaiesd
  421 nnftdttkif hdslfnsata ensmfltkia pylqvgfmpe arstislsgp gayasayydf
  481 inlqentiek tlkasdlief kfpennlsql teqeinslws fdqasakyqf ekyvrdytgg
  541 slsedngvdf nkntaldkny llnnkipsnn veeagsknyv hyiiqlqgdd isyeatcnlf
  601 sknpknsiii qrnmnesaks yflsddgesi lelnkyripe rlknkekvkv tfighgkdef
  661 ntsefarlsv dslsneissf ldtikldisp knvevnllgc nmfsydfnve etypgkllls
  721 imdkitstlp dvnknsitig anqyevrins egrkellahs gkwinkeeai msdlsskeyi
  781 ffdsidnklk aksknipgla sisediktll ldasyspdtk filnnlklni essigdyiyy
  841 eklepvknii hnsiddlide fnllenvsde lyelkklnnl dekylisfed isknnstysv
  901 rfinksnges vyvetekeif skysehitke istiknsiit dvngnlldni qldhtsqvnt
  961 lnaaffiqsl idyssnkdvl ndlstsvkvq lyaqlfstgl ntiydsiqlv nlisnavndt
1021 invlptiteg ipivstildg inlgaaikel ldehdpllkk eleakvgvla inmslsiaat
1081 vasivgigae vtifllpiag isagipslvn nelilhdkat svvnyfnhls eskkygplkt
1141 eddkilvpid dlviseidfn nnsiklgtcn ilameggsgh tvtgnidhff sspsisship
1201 slsiysaigi etenldfskk immlpnapsr vfwwetgavp glrslendgt rlldsirdly
1261 pgkfywrfya ffdyaittlk pvyedtniki kldkdtrnfi mptittneir nklsysfdga
1321 ggtyslllss ypistninls kddlwifnid nevreisien gtikkgklik dvlskidink
1381 nkliignqti dfsgdidnkd ryifltceld dkisliiein lvaksyslll sgdknylisn
1441 lsniiekint lgldskniay nytdesnnky fgaisktsqk siihykkdsk nilefyndst
1501 lefnskdfia edinvfmkdd intitgkyyv dnntdksidf sislvsknqv kvnglylnes
1561 vyssyldfvk nsdghhntsn fmnlfldnis fwklfgfeni nfvidkyftl vgktnlgyve
1621 ficdnnknid iyfgewktss skstifsgng rnvvvepiyn pdtgedists ldfsyeplyg
1681 idryinkvli apdlytslin intnyysney ypeiivlnpn tfhkkvninl dsssfeykws
1741 tegsdfilvr yleesnkkil qkirikgils ntqsfnkmsi dfkdikklsl gyimsnfksf
1801 nseneldrdh lgfkiidnkt yyydedsklv kglininnsl fyfdpiefnl vtgwqtingk
1861 kyyfdintga alisykiing khfyfnndgv mqlgvfkgpd gfeyfapant qnnniegqai
1921 vyqskfltln gkkyyfdnds kavtgwriin nekyyfnpnn aiaavglqvi dnnkyyfnpd
1981 taiiskgwqt vngsryyfdt dtaiafngyk tidgkhfyfd sdcvvkigvf stsngfeyfa
2041 pantynnnie gqaivyqskf ltlngkkyyf dnnskavtgw qtidskkyyf ntntaeaatg
2101 wqtidgkkyy fntntaeaat gwqtidgkky yfntntaias tgytiingkh fyfntdgimq
2161 igvfkgpngf eyfapantda nniegqaily qnefltlngk kyyfgsdska vtgwriinnk
2221 kyyfnpnnai aaihlctinn dkyyfsydgi lqngyitier nnfyfdanne skmvtgvfkg
2281 pngfeyfapa nthnnniegq aivyqnkflt lngkkyyfdn dskavtgwqt idgkkyyfnl
2341 ntaeaatgwq tidgkkyyfn lntaeaatgw qtidgkkyyf ntntfiastg ytsingkhfy
2401 fntdgimqig vfkgpngfey fapanthnnn iegqailyqn kfltlngkky yfgsdskavt
2461 glrtidgkky yfntntavav tgwqtingkk yyfntntsia stgytiisgk hfyfntdgim
2521 qigvfkgpdg feyfapantd anniegqair yqnrflylhd niyyfgnnsk aatgwvtidg
2581 nryyfepnta mgangyktid nknfyfrngl pqigvfkgsn gfeyfapant danniegqai
2641 ryqnrflhll gkiyyfgnns kavtgwqtin gkvyyfmpdt amaaagglfe idgviyffgv
2701 dgvkapgiyg
SEQ ID NO: 6 Clostridium difficile toxin B (TcdB)
      1 mslvnrkqle kmanvrfrtq edeyvailda leeyhnmsen tvvekylklk dinsltdiyi
    61 dtykksgrnk alkkfkeylv tevlelknnn ltpveknlhf vwiggqindt ainyinqwkd
  121 vnsdynvnvf ydsnaflint lkktvvesai ndtlesfren lndprfdynk ffrkrmeiiy
  181 dkqknfinyy kaqreenpel iiddivktyl sneyskeide lntyieesln kitqnsgndv
  241 rnfeefknge sfnlyeqelv erwnlaaasd ilrisalkei ggmyldvdml pgiqpdlfes
  301 iekpssvtvd fwemtkleai mkykeyipey tsehfdmlde evqssfesvl asksdkseif
  361 sslgdmeasp levkiafnsk giinqglisv kdsycsnliv kqienrykil nnslnpaise
  421 dndfntttnt fidsimaean adngrfmmel gkylrvgffp dvkttinlsg peayaaayqd
  481 llmfkegsmn ihlieadlrn feisktnisq steqemaslw sfddarakaq feeykrnyfe
  541 gslgeddnld fsqnivvdke yllekissla rssergyihy ivqlqgdkis yeaacnlfak
  601 tpydsvlfqk niedseiayy ynpgdgeiqe idkykipsii sdrpkikltf ighgkdefnt
  661 difagfdvds lsteieaaid lakedispks ieinllgcnm fsysinveet ypgklllkvk
  721 dkiselmpsi sqdsiivsan qyevrinseg rrelldhsge winkeesiik disskeyisf
  781 npkenkitvk sknlpelstl lqeirnnsns sdieleekvm lteceinvis nidtqiveer
  841 ieeaknitsd sinyikdefk liesisdalc dlkqqneled shfisfedis etdegfsirf
  901 inketgesif vetektifse yanhiteeis kikgtifdtv ngklvkkvnl dtthevntln
  961 aaffiqslie ynsskeslsn lsvamkvqvy aqlfstglnt itdaakvvel vstaldetid
1021 llptlseglp iiatiidgvs lgaaikelse tsdpllrqei eakigimavn lttattaiit
1081 sslgiasgfs illvplagis agipslvnne lvlrdkatkv vdyfkhvslv etegvftlld
1141 dkimmpqddl viseidfnnn sivlgkceiw rmeggsghtv tddidhffsa psityrephl
1201 siydvlevqk eeldlskdlm vlpnapnrvf awetgwtpgl rslendgtkl ldrirdnyeg
1261 efywryfafi adalittlkp ryedtnirin ldsntrsfiv piitteyire klsysfygsg
1321 gtyalslsqy nmginielse sdvwiidvdn vvrdvtiesd kikkgdlieg ilstlsieen
1381 kiilnshein fsgevngsng fvsltfsile ginaiievdl lsksykllis gelkilmlns
1441 nhiqqkidyi gfnselqkni pysfvdsegk engfingstk eglfvselpd vvliskvymd
1501 dskpsfgyys nnlkdvkvit kdnvniltgy ylkddikisl sltlqdekti klnsvhldes
1561 gvaeilkfmn rkgntntsds lmsflesmni ksifvnflqs nikfildanf iisgttsigq
1621 feficdendn iqpyfikfnt letnytlyvg nrqnmivepn ydlddsgdis stvinfsqky
1681 lygidscvnk vvispniytd einitpvyet nntypevivl danyinekin vnindlsiry
1741 vwsndgndfi lmstseenkv sqvkirfvnv fkdktlankl sfnfsdkqdv pvseiilsft
1801 psyyedglig ydlglvslyn ekfyinnfgm mvsgliyind slyyfkppvn nlitgfvtvg
1861 ddkyyfnpin ggaasigeti iddknyyfnq sgvlqtgvfs tedgfkyfap antldenleg
1921 eaidftgkli ideniyyfdd nyrgavewke ldgemhyfsp etgkafkgln qigdykyyfn
1981 sdgvmqkgfv sindnkhyfd dsgvmkvgyt eidgkhfyfa engemqigvf ntedgfkyfa
2041 hhnedlgnee geeisysgil nfnnkiyyfd dsftavvgwk dledgskyyf dedtaeayig
2101 lslindgqyy fnddgimqvg fvtindkvfy fsdsgiiesg vqniddnyfy iddngivqig
2161 vfdtsdgyky fapantvndn iygqaveysg lvrvgedvyy fgetytietg wiydmenesd
2221 kyyfnpetkk ackginlidd ikyyfdekgi mrtglisfen nnyyfnenge mqfgyinied
2281 kmfyfgedgv mqigvfntpd gfkyfahqnt ldenfegesi nytgwldlde kryyftdeyi
2341 aatgsviidg eeyyfdpdta qlvise
SEQ ID NO: 7 Anti-toxin A antibody, VH region of a humanized PA-39
(hPA-39)
Gln Val Gln Leu Val Gln Ser Gly Ala Glu Val Lys Lys Pro Gly Ala
1 5 10 15
Ser Val Lys Val Ser Cys Lys Ala Ser Gly Tyr Thr Phe Asn Asp His
                        20 25 30
Asn Ile His Trp Val Arg Gln Ala Pro Gly Gln Gly Leu Glu Trp Ile
                35 40 45
Gly Tyr Ile Tyr Pro Tyr Ile Gly Thr Thr Val Tyr Asn Gln Lys Phe
        50 55 60
Lys Ser Lys Ala Thr Leu Thr Val Asp Thr Ser Thr Ser Thr Ala Tyr
65 70 75 80
Met Glu Leu Arg Ser Leu Arg Ser Asp Asp Thr Ala Val Tyr Tyr Cys
                                85 90 95
Ser Arg Trp Gly His Arg Gly Phe Pro Tyr Trp Gly Gln Gly Thr Leu
                        100 105 110
Val Thr Val Ser Ser
                115
SEQ ID NO: 8 Anti-toxin A antibody, VH region of a humanized PA-39
(hPA-39)
Gln Val Gln Leu Val Gln Ser Gly Ala Glu Val Lys Lys Pro Gly Ala
1 5 10 15
Ser Val Lys Val Ser Cys Lys Ala Ser Gly Tyr Thr Phe Asn Asp His
                        20 25 30
Asn Ile His Trp Val Arg Gln Ala Pro Gly Gln Gly Leu Glu Trp Ile
                35 40 45
Gly Tyr Ile Tyr Pro Tyr Ile Gly Thr Thr Val Tyr Asn Gln Lys Phe
        50 55 60
Lys Ser Lys Ala Thr Leu Thr Val Asp Asn Ser Thr Ser Thr Ala Tyr
65 70 75 80
Met Glu Leu Arg Ser Leu Arg Ser Asp Asp Thr Ala Val Tyr Tyr Cys
                                85 90 95
Ser Arg Trp Gly His Arg Gly Phe Pro Tyr Trp Gly Gln Gly Thr Leu
                        100 105 110
Val Thr Val Ser Ser
                115
SEQ ID NO: 9 Anti-toxin A antibody, VL region of a humanized PA-39
(hPA-39)
Asp Ile Gln Met Thr Gln Ser Pro Ser Ser Leu Ser Ala Ser Val Gly
1 5 10 15
Asp Arg Val Thr Ile Thr Cys Lys Ala Ser Gln Asn Val Gly Thr Asn
                        20 25 30
Val Ala Trp Tyr Gln Gln Lys Pro Gly Lys Ala Pro Lys Ala Leu Ile
                35 40 45
Tyr Ser Ala Ser Tyr Arg Tyr Ser Gly Val Ser Ser Arg Phe Ser Gly
        50 55 60
Ser Gly Ser Gly Thr Asp Phe Thr Leu Thr Ile Ser Ser Leu Gln Pro
65 70 75 80
Glu Asp Phe Ala Val Tyr Tyr Cys Gln Gln Tyr Tyr Ser Tyr Pro Tyr
                                85 90 95
Thr Phe Gly Gln Gly Thr Lys Leu Glu Ile Lys
                        100 105
SEQ ID NO: 10 Anti-toxin A antibody, VL region of a humanized PA-39
(hPA-39)
Asp Ile Gln Met Thr Gln Ser Pro Ser Ser Leu Ser Ala Ser Val Gly
1 5 10 15
Asp Arg Val Thr Ile Thr Cys Lys Ala Ser Gln Asn Val Gly Thr Asn
                        20 25 30
Val Ala Trp Tyr Gln Gln Lys Pro Gly Lys Ala Pro Lys Val Leu Ile
                35 40 45
Tyr Ser Ala Ser Tyr Arg Tyr Ser Gly Val Ser Ser Arg Phe Ser Gly
        50 55 60
Ser Gly Ser Gly Thr Asp Phe Thr Leu Thr Ile Ser Ser Leu Gln Pro
65 70 75 80
Glu Asp Phe Ala Val Tyr Tyr Cys Gln Gln Tyr Tyr Ser Tyr Pro Tyr
                                85 90 95
Thr Phe Gly Gln Gly Thr Lys Leu Glu Ile Lys
                        100 105
SEQ ID NO: 11 Anti-toxin A antibody, VH region of a humanized PA-50
(hPA-50)
Gln Val Gln Leu Val Gln Ser Gly Ala Glu Val Lys Lys Pro Gly Ala
1 5 10 15
Ser Val Lys Val Ser Cys Lys Ala Ser Gly Tyr Thr Phe Thr Asp Tyr
                        20 25 30
Asn Met Asp Trp Val Arg Gln Ala Pro Gly Gln Arg Leu Glu Trp Ile
                35 40 45
Gly Asp Ile Asn Pro Lys Tyr Asp Ile Ile Gly His Asn Pro Lys Phe
        50 55 60
Met Gly Lys Ala Thr Leu Thr Val Asp Lys Ser Ala Ser Thr Ala Tyr
65 70 75 80
Met Glu Leu Ser Ser Leu Arg Ser Glu Asp Thr Ala Val Tyr Tyr Cys
                                85 90 95
Ala Arg Ser Asp Arg Gly Trp Tyr Phe Asp Val Trp Gly Gln Gly Thr
                        100 105 110
Leu Val Thr Val Ser Ser
                115
SEQ ID NO: 12 Anti-toxin A antibody, VH region of a humanized PA-50
(hPA-50)
Gln Val Gln Leu Val Gln Ser Gly Ala Glu Val Lys Lys Pro Gly Ala
1 5 10 15
Ser Val Lys Val Ser Cys Lys Ala Ser Gly Tyr Thr Phe Thr Asp Tyr
                        20 25 30
Asn Met Asp Trp Val Arg Gln Ala Pro Gly Gln Arg Leu Glu Trp Ile
                35 40 45
Gly Asp Ile Asn Pro Lys Tyr Asp Ile Ile Gly His Asn Pro Lys Phe
        50 55 60
Met Gly Lys Ala Thr Ile Thr Val Asp Lys Ser Ala Ser Thr Ala Tyr
65 70 75 80
Met Glu Leu Ser Ser Leu Arg Ser Glu Asp Thr Ala Val Tyr Tyr Cys
                                85 90 95
Ala Arg Ser Asp Arg Gly Trp Tyr Phe Asp Val Trp Gly Gln Gly Thr
                        100 105 110
Leu Val Thr Val Ser Ser
                115
SEQ ID NO: 13 Anti-toxin A antibody, VL region of a humanized PA-50
(hPA-50)
Glu Ile Val Leu Thr Gln Ser Pro Ala Thr Leu Ser Leu Ser Pro Gly
1 5 10 15
Glu Arg Ala Thr Leu Ser Cys Arg Ala Ser Ser Ser Val Asn Tyr Met
                        20 25 30
Asn Trp Tyr Gln Gln Lys Pro Gly Gln Ala Pro Arg Pro Arg Ile Tyr
                35 40 45
Ala Thr Ser Asn Leu Ala Ser Gly Val Pro Ala Arg Phe Ser Gly Ser
        50 55 60
Gly Ser Gly Thr Asp Tyr Thr Leu Thr Ile Ser Ser Leu Glu Pro Glu
65 70 75 80
Asp Phe Ala Val Tyr Tyr Cys Gln Gln Trp Ser Ser Arg Thr Phe Gly
                                85 90 95
Gly Gly Thr Lys Val Glu Ile Lys
                        100
SEQ ID NO: 14 Anti-toxin B antibody, VH region of a humanized PA-41
(hPA-41)
Gln Val Gln Leu Val Gln Ser Gly Ala Glu Val Lys Lys Pro Gly Ala
1 5 10 15
Ser Val Lys Val Ser Cys Lys Ala Ser Gly Tyr Pro Phe Thr Asn Tyr
                        20 25 30
Phe Met His Trp Val Arg Gln Ala Pro Gly Gln Arg Leu Glu Trp Ile
                35 40 45
Gly Arg Ile Asn Pro Tyr Asn Gly Ala Thr Ser Tyr Ser Leu Asn Phe
        50 55 60
Arg Asp Lys Ala Thr Leu Thr Leu Asp Lys Ser Ala Ser Thr Ala Tyr
65 70 75 80
Met Glu Leu Ser Ser Leu Arg Ser Glu Asp Thr Ala Val Tyr Tyr Cys
                                85 90 95
Ala Arg Ser Thr Ile Thr Ser Pro Leu Leu Asp Phe Trp Gly Gln Gly
                        100 105 110
Thr Leu Val Thr Val Ser Ser
                115
SEQ ID NO: 15 Anti-toxin B antibody, VH region of a humanized PA-41
(hPA-41)
Gln Val Gln Leu Val Gln Ser Gly Ala Glu Val Lys Lys Pro Gly Ala
1 5 10 15
Ser Val Lys Val Ser Cys Lys Ala Ser Gly Tyr Pro Phe Thr Asn Tyr
                        20 25 30
Phe Met His Trp Val Arg Gln Ala Pro Gly Gln Arg Leu Glu Trp Ile
                35 40 45
Gly Arg Ile Asn Pro Tyr Asn Gly Ala Thr Ser Tyr Ser Leu Asn Phe
        50 55 60
Arg Asp Lys Ala Thr Ile Thr Leu Asp Lys Ser Ala Ser Thr Ala Tyr
65 70 75 80
Met Glu Leu Ser Ser Leu Arg Ser Glu Asp Thr Ala Val Tyr Tyr Cys
                                85 90 95
Ala Arg Ser Thr Ile Thr Ser Pro Leu Leu Asp Phe Trp Gly Gln Gly
                        100 105 110
Thr Leu Val Thr Val Ser Ser
                115
SEQ ID NO: 16 Anti-toxin B antibody, VL region of a humanized PA-41
(hPA-41)
Glu Ile Val Leu Thr Gln Ser Pro Ala Thr Leu Ser Leu Ser Pro Gly
1 5 10 15
Glu Arg Ala Thr Leu Ser Cys Arg Ala Ser Gln Ser Val Gly Thr Ser
                        20 25 30
Ile His Trp Tyr Gln Gln Lys Pro Gly Gln Ala Pro Arg Leu Leu Ile
                35 40 45
Lys Phe Ala Ser Glu Ser Ile Ser Gly Ile Pro Ala Arg Phe Ser Gly
        50 55 60
Ser Gly Ser Gly Thr Asp Phe Thr Leu Thr Ile Ser Ser Leu Glu Pro
65 70 75 80
Glu Asp Phe Ala Val Tyr Tyr Cys Gln Gln Ser Asn Lys Trp Pro Phe
                                85 90 95
Thr Phe Gly Gln Gly Thr Lys Leu Glu Ile Lys
                        100 105
SEQ ID NO: 17 Anti-toxin A antibody, heavy chain
Met Glu Trp Ser Gly Val Phe Ile Phe Leu Leu Ser Val Thr Ala Gly
1 5 10 15
Val His Ser Gln Val Gln Leu Val Gln Ser Gly Ala Glu Val Lys Lys
                        20 25 30
Pro Gly Ala Ser Val Lys Val Ser Cys Lys Ala Ser Gly Tyr Thr Phe
                35 40 45
Thr Asp Tyr Asn Met Asp Trp Val Arg Gln Ala Pro Gly Gln Arg Leu
        50 55 60
Glu Trp Ile Gly Asp Ile Asn Pro Lys Tyr Asp Ile Ile Gly His Asn
65 70 75 80
Pro Lys Phe Met Gly Lys Ala Thr Ile Thr Val Asp Lys Ser Ala Ser
                                85 90 95
Thr Ala Tyr Met Glu Leu Ser Ser Leu Arg Ser Glu Asp Thr Ala Val
                        100 105 110
Tyr Tyr Cys Ala Arg Ser Asp Arg Gly Trp Tyr Phe Asp Val Trp Gly
                115 120 125
Gln Gly Thr Leu Val Thr Val Ser Ser Ala Ser Thr Lys Gly Pro Ser
        130 135 140
Val Phe Pro Leu Ala Pro Ser Ser Lys Ser Thr Ser Gly Gly Thr Ala
145 150 155 160
Ala Leu Gly Cys Leu Val Lys Asp Tyr Phe Pro Glu Pro Val Thr Val
                                165 170 175
Ser Trp Asn Ser Gly Ala Leu Thr Ser Gly Val His Thr Phe Pro Ala
                        180 185 190
Val Leu Gln Ser Ser Gly Leu Tyr Ser Leu Ser Ser Val Val Thr Val
                195 200 205
Pro Ser Ser Ser Leu Gly Thr Gln Thr Tyr Ile Cys Asn Val Asn His
        210 215 220
Lys Pro Ser Asn Thr Lys Val Asp Lys Arg Val Glu Pro Lys Ser Cys
225 230 235 240
Asp Lys Thr His Thr Cys Pro Pro Cys Pro Ala Pro Glu Leu Leu Gly
                                245 250 255
Gly Pro Ser Val Phe Leu Phe Pro Pro Lys Pro Lys Asp Thr Leu Met
                        260 265 270
Ile Ser Arg Thr Pro Glu Val Thr Cys Val Val Val Asp Val Ser His
                275 280 285
Glu Asp Pro Glu Val Lys Phe Asn Trp Tyr Val Asp Gly Val Glu Val
        290 295 300
His Asn Ala Lys Thr Lys Pro Arg Glu Glu Gln Tyr Asn Ser Thr Tyr
305 310 315 320
Arg Val Val Ser Val Leu Thr Val Leu His Gln Asp Trp Leu Asn Gly
                                325 330 335
Lys Glu Tyr Lys Cys Lys Val Ser Asn Lys Ala Leu Pro Ala Pro Ile
                        340 345 350
Glu Lys Thr Ile Ser Lys Ala Lys Gly Gln Pro Arg Glu Pro Gln Val
                355 360 365
Tyr Thr Leu Pro Pro Ser Arg Glu Glu Met Thr Lys Asn Gln Val Ser
        370 375 380
Leu Thr Cys Leu Val Lys Gly Phe Tyr Pro Ser Asp Ile Ala Val Glu
385 390 395 400
Trp Glu Ser Asn Gly Gln Pro Glu Asn Asn Tyr Lys Thr Thr Pro Pro
                                405 410 415
Val Leu Asp Ser Asp Gly Ser Phe Phe Leu Tyr Ser Lys Leu Thr Val
                        420 425 430
Asp Lys Ser Arg Trp Gln Gln Gly Asn Val Phe Ser Cys Ser Val Met
                435 440 445
His Glu Ala Leu His Asn His Tyr Thr Gln Lys Ser Leu Ser Leu Ser
        450 455 460
Pro Gly Lys
465
SEQ ID NO: 18 Anti-toxin A antibody, light chain
Met Asp Phe Gln Val Gln Ile Phe Ser Phe Leu Leu Ile Ser Ala Ser
1 5 10 15
Val Ile Met Ser Arg Gly Glu Ile Val Leu Thr Gln Ser Pro Ala Thr
                        20 25 30
Leu Ser Leu Ser Pro Gly Glu Arg Ala Thr Leu Ser Cys Arg Ala Ser
                35 40 45
Ser Ser Val Asn Tyr Met Asn Trp Tyr Gln Gln Lys Pro Gly Gln Ala
        50 55 60
Pro Arg Pro Arg Ile Tyr Ala Thr Ser Asn Leu Ala Ser Gly Val Pro
65 70 75 80
Ala Arg Phe Ser Gly Ser Gly Ser Gly Thr Asp Tyr Thr Leu Thr Ile
                                85 90 95
Ser Ser Leu Glu Pro Glu Asp Phe Ala Val Tyr Tyr Cys Gln Gln Trp
                        100 105 110
Ser Ser Arg Thr Phe Gly Gly Gly Thr Lys Leu Glu Ile Lys Arg Thr
                115 120 125
Val Ala Ala Pro Ser Val Phe Ile Phe Pro Pro Ser Asp Glu Gln Leu
        130 135 140
Lys Ser Gly Thr Ala Ser Val Val Cys Leu Leu Asn Asn Phe Tyr Pro
145 150 155 160
Arg Glu Ala Lys Val Gln Trp Lys Val Asp Asn Ala Leu Gln Ser Gly
                                165 170 175
Asn Ser Gln Glu Ser Val Thr Glu Gln Asp Ser Lys Asp Ser Thr Tyr
                        180 185 190
Ser Leu Ser Ser Thr Leu Thr Leu Ser Lys Ala Asp Tyr Glu Lys His
                195 200 205
Lys Val Tyr Ala Cys Glu Val Thr His Gln Gly Leu Ser Ser Pro Val
        210 215 220
Thr Lys Ser Phe Asn Arg Gly Glu Cys
225 230
SEQ ID NO: 19 Anti-toxin A antibody, heavy chain
Met Glu Trp Ser Gly Val Phe Ile Phe Leu Leu Ser Val Thr Ala Gly
1 5 10 15
Val His Ser Gln Val Gln Leu Val Gln Ser Gly Ala Glu Val Lys Lys
                        20 25 30
Pro Gly Ala Ser Val Lys Val Ser Cys Lys Ala Ser Gly Tyr Thr Phe
                35 40 45
Asn Asp His Asn Ile His Trp Val Arg Gln Ala Pro Gly Gln Gly Leu
        50 55 60
Glu Trp Ile Gly Tyr Ile Tyr Pro Tyr Ile Gly Thr Thr Val Tyr Asn
65 70 75 80
Gln Lys Phe Lys Ser Lys Ala Thr Leu Thr Val Asp Thr Ser Thr Ser
                                85 90 95
Thr Ala Tyr Met Glu Leu Arg Ser Leu Arg Ser Asp Asp Thr Ala Val
                        100 105 110
Tyr Tyr Cys Ser Arg Trp Gly His Arg Gly Phe Pro Tyr Trp Gly Gln
                115 120 125
Gly Thr Leu Val Thr Val Ser Ser Ala Ser Thr Lys Gly Pro Ser Val
        130 135 140
Phe Pro Leu Ala Pro Ser Ser Lys Ser Thr Ser Gly Gly Thr Ala Ala
145 150 155 160
Leu Gly Cys Leu Val Lys Asp Tyr Phe Pro Glu Pro Val Thr Val Ser
                                165 170 175
Trp Asn Ser Gly Ala Leu Thr Ser Gly Val His Thr Phe Pro Ala Val
                        180 185 190
Leu Gln Ser Ser Gly Leu Tyr Ser Leu Ser Ser Val Val Thr Val Pro
                195 200 205
Ser Ser Ser Leu Gly Thr Gln Thr Tyr Ile Cys Asn Val Asn His Lys
        210 215 220
Pro Ser Asn Thr Lys Val Asp Lys Arg Val Gly Glu Arg Pro Ala Gln
225 230 235 240
Gly Gly Arg Val Ser Ala Gly Ser Gln Ala Gln Arg Ser Cys Leu Asp
                                245 250 255
Ala Ser Arg Leu Cys Ser Pro Ser Pro Gly Gln Gln Gly Arg Pro Arg
                        260 265 270
Leu Pro Leu His Pro Glu Ala Ser Ala Arg Pro Thr His Ala Gln Gly
                275 280 285
Glu Gly Leu Leu Ala Phe Ser Pro Gly Ser Gly Gln Ala Gln Ala Arg
        290 295 300
Cys Pro Glu Pro Lys Ser Cys Asp Lys Thr His Thr Cys Pro Pro Cys
305 310 315 320
Pro Ala Pro Glu Leu Leu Gly Gly Pro Ser Val Phe Leu Phe Pro Pro
                                325 330 335
Lys Pro Lys Asp Thr Leu Met Ile Ser Arg Thr Pro Glu Val Thr Cys
                        340 345 350
Val Val Val Asp Val Ser His Glu Asp Pro Glu Val Lys Phe Asn Trp
                355 360 365
Tyr Val Asp Gly Val Glu Val His Asn Ala Lys Thr Lys Pro Arg Glu
        370 375 380
Glu Gln Tyr Asn Ser Thr Tyr Arg Val Val Ser Val Leu Thr Val Leu
385 390 395 400
His Gln Asp Trp Leu Asn Gly Lys Glu Tyr Lys Cys Lys Val Ser Asn
                                405 410 415
Lys Ala Leu Pro Ala Pro Ile Glu Lys Thr Ile Ser Lys Ala Lys Gly
                        420 425 430
Gln Pro Arg Glu Pro Gln Val Tyr Thr Leu Pro Pro Ser Arg Glu Glu
                435 440 445
Met Thr Lys Asn Gln Val Ser Leu Thr Cys Leu Val Lys Gly Phe Tyr
        450 455 460
Pro Ser Asp Ile Ala Val Glu Trp Glu Ser Asn Gly Gln Pro Glu Asn
465 470 475 480
Asn Tyr Lys Thr Thr Pro Pro Val Leu Asp Ser Asp Gly Ser Phe Phe
                                485 490 495
Leu Tyr Ser Lys Leu Thr Val Asp Lys Ser Arg Trp Gln Gln Gly Asn
                        500 505 510
Val Phe Ser Cys Ser Val Met His Glu Ala Leu His Asn His Tyr Thr
                515 520 525
Gln Lys Ser Leu Ser Leu Ser Pro Gly Lys
        530 535
SEQ ID NO: 20 Anti-toxin A antibody, light chain
Met Glu Ser Gln Thr Gln Val Phe Val Tyr Met Leu Leu Trp Leu Ser
1 5 10 15
Gly Val Asp Gly Asp Ile Gln Met Thr Gln Ser Pro Ser Ser Leu Ser
                        20 25 30
Ala Ser Val Gly Asp Arg Val Thr Ile Thr Cys Lys Ala Ser Gln Asn
                35 40 45
Val Gly Thr Asn Val Ala Trp Tyr Gln Gln Lys Pro Gly Lys Ala Pro
        50 55 60
Lys Ala Leu Ile Tyr Ser Ala Ser Tyr Arg Tyr Ser Gly Val Ser Ser
65 70 75 80
Arg Phe Ser Gly Ser Gly Ser Gly Thr Asp Phe Thr Leu Thr Ile Ser
                                85 90 95
Ser Leu Gln Pro Glu Asp Phe Ala Val Tyr Tyr Cys Gln Gln Tyr Tyr
                        100 105 110
Ser Tyr Pro Tyr Thr Phe Gly Gln Gly Thr Lys Leu Glu Ile Lys Arg
                115 120 125
Thr Val Ala Ala Pro Ser Val Phe Ile Phe Pro Pro Ser Asp Glu Gln
        130 135 140
Leu Lys Ser Gly Thr Ala Ser Val Val Cys Leu Leu Asn Asn Phe Tyr
145 150 155 160
Pro Arg Glu Ala Lys Val Gln Trp Lys Val Asp Asn Ala Leu Gln Ser
                                165 170 175
Gly Asn Ser Gln Glu Ser Val Thr Glu Gln Asp Ser Lys Asp Ser Thr
                        180 185 190
Tyr Ser Leu Ser Ser Thr Leu Thr Leu Ser Lys Ala Asp Tyr Glu Lys
                195 200 205
His Lys Val Tyr Ala Cys Glu Val Thr His Gln Gly Leu Ser Ser Pro
        210 215 220
Val Thr Lys Ser Phe Asn Arg Gly Glu Cys
225 230
SEQ ID NO: 21 Anti-toxin B antibody, heavy chain
Met Gly Trp Ser Trp Ile Phe Leu Phe Leu Leu Ser Gly Thr Ala Gly
1 5 10 15
Gly Leu Ser Gln Val Gln Leu Val Gln Ser Gly Ala Glu Val Lys Lys
                        20 25 30
Pro Gly Ala Ser Val Lys Val Ser Cys Lys Ala Ser Gly Tyr Pro Phe
                35 40 45
Thr Asn Tyr Phe Met His Trp Val Arg Gln Ala Pro Gly Gln Arg Leu
        50 55 60
Glu Trp Ile Gly Arg Ile Asn Pro Tyr Asn Gly Ala Thr Ser Tyr Ser
65 70 75 80
Leu Asn Phe Arg Asp Lys Ala Thr Ile Thr Leu Asp Lys Ser Ala Ser
                                85 90 95
Thr Ala Tyr Met Glu Leu Ser Ser Leu Arg Ser Glu Asp Thr Ala Val
                        100 105 110
Tyr Tyr Cys Ala Arg Ser Thr Ile Thr Ser Pro Leu Leu Asp Phe Trp
                115 120 125
Gly Gln Gly Thr Leu Val Thr Val Ser Ser Ala Ser Thr Lys Gly Pro
        130 135 140
Ser Val Phe Pro Leu Ala Pro Ser Ser Lys Ser Thr Ser Gly Gly Thr
145 150 155 160
Ala Ala Leu Gly Cys Leu Val Lys Asp Tyr Phe Pro Glu Pro Val Thr
                                165 170 175
Val Ser Trp Asn Ser Gly Ala Leu Thr Ser Gly Val His Thr Phe Pro
                        180 185 190
Ala Val Leu Gln Ser Ser Gly Leu Tyr Ser Leu Ser Ser Val Val Thr
                195 200 205
Val Pro Ser Ser Ser Leu Gly Thr Gln Thr Tyr Ile Cys Asn Val Asn
        210 215 220
His Lys Pro Ser Asn Thr Lys Val Asp Lys Arg Val Gly Glu Arg Pro
225 230 235 240
Ala Gln Gly Gly Arg Val Ser Ala Gly Ser Gln Ala Gln Arg Ser Cys
                                245 250 255
Leu Asp Ala Ser Arg Leu Cys Ser Pro Ser Pro Gly Gln Gln Gly Arg
                        260 265 270
Pro Arg Leu Pro Leu His Pro Glu Ala Ser Ala Arg Pro Thr His Ala
                275 280 285
Gln Gly Glu Gly Leu Leu Ala Phe Ser Pro Gly Ser Gly Gln Ala Gln
        290 295 300
Ala Arg Cys Pro Glu Pro Lys Ser Cys Asp Lys Thr His Thr Cys Pro
305 310 315 320
Pro Cys Pro Ala Pro Glu Leu Leu Gly Gly Pro Ser Val Phe Leu Phe
                                325 330 335
Pro Pro Lys Pro Lys Asp Thr Leu Met Ile Ser Arg Thr Pro Glu Val
                        340 345 350
Thr Cys Val Val Val Asp Val Ser His Glu Asp Pro Glu Val Lys Phe
                355 360 365
Asn Trp Tyr Val Asp Gly Val Glu Val His Asn Ala Lys Thr Lys Pro
        370 375 380
Arg Glu Glu Gln Tyr Asn Ser Thr Tyr Arg Val Val Ser Val Leu Thr
385 390 395 400
Val Leu His Gln Asp Trp Leu Asn Gly Lys Glu Tyr Lys Cys Lys Val
                                405 410 415
Ser Asn Lys Ala Leu Pro Ala Pro Ile Glu Lys Thr Ile Ser Lys Ala
                        420 425 430
Lys Gly Gln Pro Arg Glu Pro Gln Val Tyr Thr Leu Pro Pro Ser Arg
                435 440 445
Glu Glu Met Thr Lys Asn Gln Val Ser Leu Thr Cys Leu Val Lys Gly
        450 455 460
Phe Tyr Pro Ser Asp Ile Ala Val Glu Trp Glu Ser Asn Gly Gln Pro
465 470 475 480
Glu Asn Asn Tyr Lys Thr Thr Pro Pro Val Leu Asp Ser Asp Gly Ser
                                485 490 495
Phe Phe Leu Tyr Ser Lys Leu Thr Val Asp Lys Ser Arg Trp Gln Gln
                        500 505 510
Gly Asn Val Phe Ser Cys Ser Val Met His Glu Ala Leu His Asn His
                515 520 525
Tyr Thr Gln Lys Ser Leu Ser Leu Ser Pro Gly Lys
        530 535 540
SEQ ID NO: 22 Anti-toxin B antibody, light chain
Met Ser Val Pro Thr Gln Val Leu Gly Leu Leu Leu Leu Trp Leu Thr
1 5 10 15
Asp Ala Arg Cys Glu Ile Val Leu Thr Gln Ser Pro Ala Thr Leu Ser
                        20 25 30
Leu Ser Pro Gly Glu Arg Ala Thr Leu Ser Cys Arg Ala Ser Gln Ser
                35 40 45
Val Gly Thr Ser Ile His Trp Tyr Gln Gln Lys Pro Gly Gln Ala Pro
        50 55 60
Arg Leu Leu Ile Lys Phe Ala Ser Glu Ser Ile Ser Gly Ile Pro Ala
65 70 75 80
Arg Phe Ser Gly Ser Gly Ser Gly Thr Asp Phe Thr Leu Thr Ile Ser
                                85 90 95
Ser Leu Glu Pro Glu Asp Phe Ala Val Tyr Tyr Cys Gln Gln Ser Asn
                        100 105 110
Lys Trp Pro Phe Thr Phe Gly Gln Gly Thr Lys Leu Glu Ile Lys Arg
                115 120 125
Thr Val Ala Ala Pro Ser Val Phe Ile Phe Pro Pro Ser Asp Glu Gln
        130 135 140
Leu Lys Ser Gly Thr Ala Ser Val Val Cys Leu Leu Asn Asn Phe Tyr
145 150 155 160
Pro Arg Glu Ala Lys Val Gln Trp Lys Val Asp Asn Ala Leu Gln Ser
                                165 170 175
Gly Asn Ser Gln Glu Ser Val Thr Glu Gln Asp Ser Lys Asp Ser Thr
                        180 185 190
Tyr Ser Leu Ser Ser Thr Leu Thr Leu Ser Lys Ala Asp Tyr Glu Lys
                195 200 205
His Lys Val Tyr Ala Cys Glu Val Thr His Gln Gly Leu Ser Ser Pro
        210 215 220
Val Thr Lys Ser Phe Asn Arg Gly Glu Cys
225 230
```

1. A method of treating a C. difficile infection or C. difficile-associated disease in a subject, the method comprising administering to the subject a combination of an anti-C. difficile toxin A antibody and an anti-C. difficile toxin B antibody comprising an alteration that increases the half-life of one or both antibodies relative to anti-C. difficile toxin A and B antibodies lacking the alteration.

2. A method of treating a C. difficile infection or C. difficile-associated disease in a subject, the method comprising administering to the subject a combination of an anti-C. difficile toxin A antibody and an anti-C. difficile toxin B antibody and vancomycin, to thereby reduce the dose or dose frequency of vancomycin relative to a reference dose or dose frequency.

3. The method of claim 2, wherein one or both antibodies has an alteration that increases its half-life relative to anti-C. difficile toxin A and B antibodies lacking the alteration.

4. The method of claim 1, wherein the alteration is any one or more of 252Y, 254T, or 256E.

5. The method of claim 1, wherein the alteration is conjugation to polyethylene glycol (PEG) or conjugation to albumin.

6. The method of claim 1, wherein the anti-toxin A antibody is selected from the group consisting of:
(a) an anti-C. difficile toxin A antibody having:
(i) a heavy chain comprising the sequence of SEQ ID NO: 1; and/or
(ii) a light chain comprising the sequence of SEQ ID NO: 2; and
(b) PA50-YTE.

7. (canceled)

8. (canceled)

9. The method of claim 1, wherein the anti-toxin B antibody is selected from the group consisting of:
(a) an anti-C. difficile toxin B antibody having:
(i) a heavy chain comprising the sequence of SEQ ID NO: 3; and/or
(ii) a light chain comprising the sequence of SEQ ID NO:4; and
(b) PA41-YTE.

10. (canceled)

11. (canceled)

12. The method of claim 1, wherein the combination of the antibodies is PA50YTE/PA41YTE COMBINATION.

13. The method of claim 12, wherein PA50YTE/PA41YTE COMBINATION is administered in a single dose.

14. The method of claim 1, further comprising administering an antibiotic.

15. The method of claim 2, wherein the vancomycin is administered orally or intravenously.

16. The method of claim 2, wherein the reference dose and dose frequency is intravenous administration of vancomycin at 15-20 mg/kg, 2-3 times daily.

17. The method of claim 2, wherein the reference dose and dose frequency is oral administration at 125 mg, 3-4 times daily.

18. The method of claim 1, wherein the method reduces the time to C. difficile reinfection.

19. The method of claim 1, wherein C. difficile toxin A and/or toxin B are neutralized.

20. The method of claim 1, wherein the method enhances microbiome restoration, reduces microbiome dysbiosis, and/or reduces intestinal damage in the subject.

21. The method of claim 1, wherein the method enhances microbiome restoration and/or reduces microbiome dysbiosis relative to an antibiotic therapy.

22. The method of claim 2, wherein one or both antibodies has an alteration that increases its half-life relative to anti-C. difficile toxin A and B antibodies lacking the alteration and wherein the alteration is any one or more of 252Y, 254T, or 256E.

23. The method of claim 2, wherein the anti-toxin A antibody is selected from the group consisting of:
(a) an anti-toxin A antibody having:
(i) a heavy chain comprising the sequence of SEQ ID NO: 1; and/or
(ii) a light chain comprising the sequence SEQ ID NO: 2; and
(b) PA50-YTE.

24. The method of claim 2, wherein the anti-toxin B antibody is selected from the group consisting of:
(a) an anti-toxin B antibody having:
(i) a heavy chain comprising the sequence of SEQ ID NO: 3; and/or
(ii) a light chain comprising the sequence of SEQ ID NO: 4; and
(b) PA41-YTE.
